(19) United States
(12) Patent Application Publication
(10) Pub. No.: US 2008/0194954 A1
(43) Pub. Date: Aug. 14, 2008

(54) Ultrasound Device and Method Using Same

(75) Inventors: Evan C. Unger, Tucson, AZ (US); Terry O. Matsunaga, Tucson, AZ (US); Reena Zutshi, Tucson, AZ (US)

Correspondence Address: DALE F. REGELMAN, QUARLES & BRADY, LLP, ONE SOUTH CHURCH AVENUE AVE, STE. 1700, TUCSON, AZ, 85701-1621, US

(73) Assignee: ImaRx Therapeutics, Inc., Tucson, AZ (US)

(21) Appl. No.: 11/570,227

(22) PCT Filed: Jun. 10, 2005
(86) PCT No.: PCT/US2005/020837

§ 371 Date: Oct. 13, 2007

Related U.S. Application Data
(60) Provisional application No. 60/578,954, filed on Jun. 10, 2004.

(51) Int. Cl. A61B 8/13; A61N 7/00

(52) U.S. Cl. 600/439; 601/2

(57) ABSTRACT

An ultrasound energy emitting apparatus is disclosed. The ultrasound energy emitting apparatus comprises a hand-held enclosure and a plurality of ultrasound transducers disposed on that enclosure, or disposed within and extending outwardly from the enclosure. The plurality of ultrasound transducers can be operated simultaneously, or in a programmed fashion whereunder one or more of, but fewer than all, of the transducers emit ultrasound energy at one time.

### CROSS REFERENCE TO RELATED CASES

This application claims priority from a U.S. Provisional Application having Ser. No. 60/578,954 filed Jun. 10, 2004.

### FIELD OF THE INVENTION

Applicants' invention relates to an ultrasound emitting device, and a method using same.

### BACKGROUND OF THE INVENTION

Thrombosis, the formation and development of a blood clot or thrombus within the vascular system, can be life threatening. The thrombus can block a vessel and stop blood supply to an organ or other body part. If detached, the thrombus can become an embolus and occlude a vessel distant from the original site.

Dissolution of thrombus using ultrasound is known in the art. Further, the ability of microbubbles to potentiate ultrasound-induced thrombolysis is known. The bubbles are destroyed by the ultrasound and the energy is released into the clot.

What is needed, however, is an ultrasound emitting device which can better direct the emitted ultrasound energy to the occlusion site, thereby enhancing the effectiveness of the ultrasound energy/microbubble interaction. Applicants' apparatus provides such an ultrasound emitting device.

Prior art therapeutic ultrasound emitting devices comprise a single ultrasound transducer. In contrast, Applicants' apparatus comprises a plurality of ultrasound transducers. Applicants' plurality of ultrasound transducers can be operated simultaneously, or in a programmed fashion whereunder one or more of, but fewer than all, of the transducers emit ultrasound energy at one time.

### SUMMARY OF THE INVENTION

Applicants' invention comprises an ultrasound energy emitting apparatus. Applicants' ultrasound energy emitting apparatus comprises a hand-held enclosure and a plurality of ultrasound transducers disposed on that enclosure, or disposed within and extending outwardly from the enclosure. Applicants' plurality of ultrasound transducers can be operated simultaneously, or in a programmed fashion whereunder one or more of, but fewer than all, of the transducers emit ultrasound energy at one time. Applicants' invention further comprises a method using Applicants' apparatus to treat a patient having an occlusion lodged in a blood vessel.

### BRIEF DESCRIPTION OF THE DRAWINGS

The invention will be better understood from a reading of the following detailed description taken in conjunction with the drawings in which like reference designators are used to designate like elements, and in which:

FIG. 1A is a perspective view of Applicants' hand-held ultrasound emitting device;

FIG. 1B is a side view of the device of FIG. 1;

FIG. 1C is a perspective view of the device of FIG. 1 showing a housing portion and a bottom portion;

FIG. 2A is a perspective view of an embodiment of Applicants' hand-held ultrasound emitting device comprising a bottom portion comprising two offset planar members;

FIG. 2B is a perspective view of the bottom portion of FIG. 2A;

FIG. 2C is a side view of the bottom portion of FIG. 2A;

FIG. 3A is a perspective view of an embodiment of Applicants' hand-held ultrasound emitting device comprising a bottom portion comprising four offset planar members;

FIG. 3B is a side view of the bottom portion of FIG. 3A;

FIG. 4A is a block diagram showing one embodiment of Applicants' sound head matrix;

FIG. 4B is a side view of one embodiment of the sound head matrix of FIG. 4A;

FIG. 4C is a side view of a second embodiment of the sound head matrix of FIG. 4A;

FIG. 5A is a block diagram showing a second embodiment of Applicants' sound head matrix;

FIG. 5B is a side view of one embodiment of the sound head matrix of FIG. 5A;

FIG. 5C is a side view of a second embodiment of the sound head matrix of FIG. 5A;

FIG. 6 is a perspective view showing an external controller and power source for Applicants' hand-held ultrasound emitting device;

FIG. 7A is a perspective view showing an embodiment of Applicants' hand-held ultrasound emitting device comprising an internal controller;

FIG. 7B is a perspective view showing the device of FIG. 7A in combination with an integrated input/output element;

FIG. 8A is a block diagram showing an embodiment of Applicants' hand-held ultrasound emitting device which further comprises a diagnostic ultrasound transceiver;

FIG. 8B is a perspective view of the device of FIG. 8A further comprising an internal controller;

FIG. 8C is a perspective view of the device of FIG. 8B further comprising an integrated input/output element;

FIG. 8D is a perspective view of the device of FIG. 8C as that device is positioned with respect to venus blood flow;

FIG. 8E is a perspective view of the device of FIG. 8A further comprising an integrated controller comprising an auto-detect function;

FIG. 9 is a flow chart summarizing the steps of Applicants' method using Applicants' hand-held ultrasound emitting device;

FIG. 10 is a chart reciting the depth from skin surface of certain veins for a first patient;

FIG. 11 is a chart reciting the depth from skin surface of certain veins for a second patient;

FIG. 12 is a chart reciting the depth from skin surface of certain veins for a third patient;

FIG. 13 is a perspective view showing an occlusion site in a blood vessel;

FIG. 14A is a block diagram showing the ultrasound emissions from an offset sound head matrix comprising two planar assemblies;

FIG. 14B shows the convergence point for the device of FIG. 14A;

FIG. 15A is a block diagram showing the ultrasound emissions from an offset sound head matrix comprising three planar assemblies;

FIG. 15B shows the convergence point for the device of FIG. 15A;

FIG. 16A is a block diagram showing the ultrasound emissions from an offset sound head matrix comprising four planar assemblies;

FIG. 16B shows the convergence point for the device of FIG. 16A;

FIG. 17 is a bottom view of Applicants' hand-held device showing certain attachment means used to attach the device to a patient's extremity;

FIG. 18 is a side view showing Applicants' hand-held ultrasound emitting device and an ultrasound coupling medium positioned on the skin surface over an occlusion site.

### DETAILED DESCRIPTION OF THE PREFERRED EMBODIMENTS

This invention is described in preferred embodiments in the following description with reference to the Figures, in which like numbers represent the same or similar elements. The invention will be described as embodied in a hand-held ultrasound emitting device having a curved top portion. The following description of Applicant's apparatus, and method using that apparatus, is not meant, however, to limit Applicant's invention to hand-held devices having a curved top, or to only hand-held devices, as the invention herein can be applied to devices to production of ultrasound energy in general.

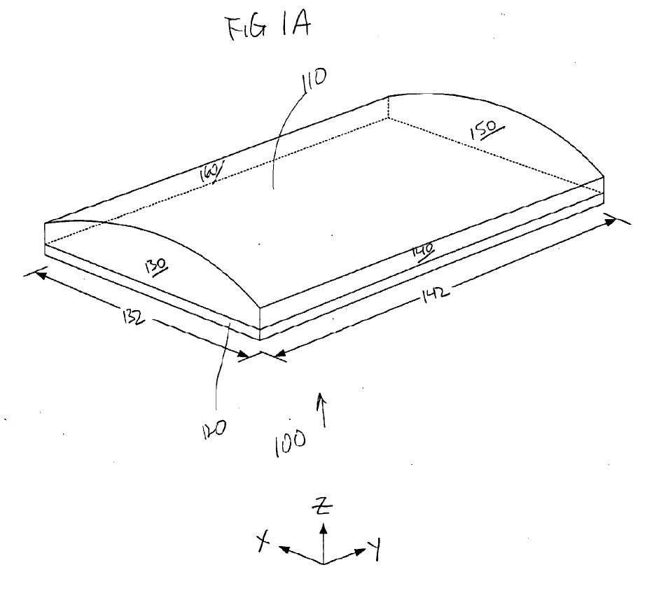

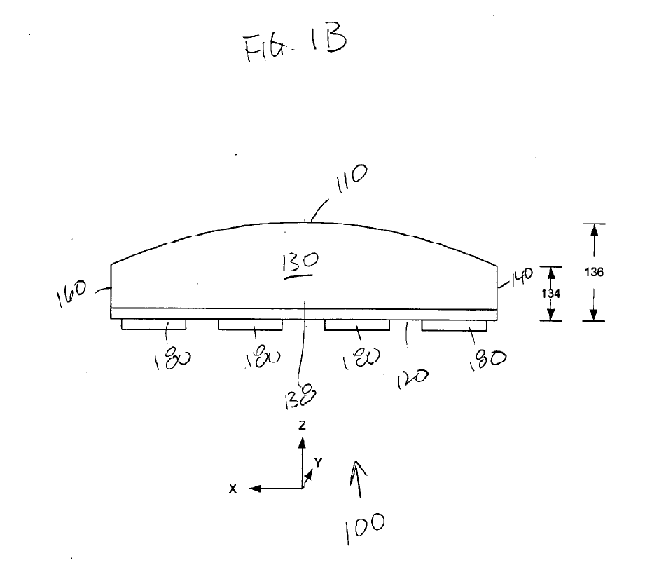

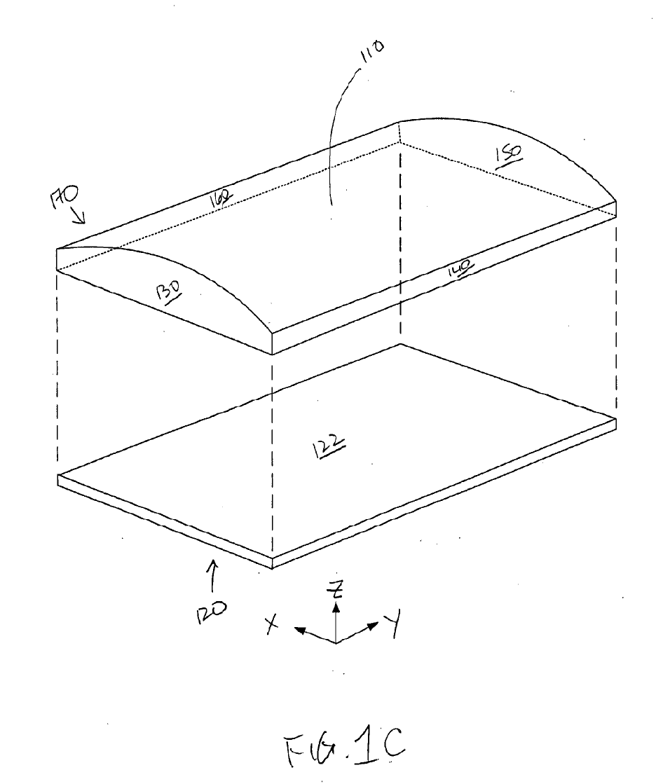

Referring to FIG. 1A, Applicants' hand-held ultrasound emitting device 100 comprises a top 110, bottom 120, and sides 130, 140, 150, and 160. In certain embodiments, top 110 and sides 130, 140, 150, and 160, are formed from one or more rigid materials, including wood, metal, plastic, and combinations thereof. In certain embodiments, top 110, and sides 130, 140, 150, and 160, are separately formed, and subsequent attached to one another as shown in FIG. 1 using conventional attachment methods, including welding, sonic welding, plastic welding, adhesive bonding, mechanical attachment, and the like.

Sides 140 and 160 have dimension 142 in the Y direction. In certain embodiments, dimension 142 is between about 10 cm and about 50 cm. Sides 130 and 150 have dimension 132 in the X direction. In certain embodiments, dimension 132 is between about 5 cm and about 25 cm.

FIG. 1B is a side view of apparatus 100. Apparatus 100 includes a plurality of therapeutic ultrasound transducers 180 disposed on, or through, bottom 120. By “therapeutic ultrasound transducer,” Applicants mean a device that is capable of operating at between a 0.1 percent and a 100 percent duty cycle, and that emits therapeutic ultrasound energy. By “therapeutic ultrasound energy,” Applicants mean sound waves having a frequency between about 150 kilohertz and about 10 megahertz or higher, and a power level between about 0.1 watt/cm² and about 30 watts/cm². In certain embodiments, when operated continuously, the output power for each of the plurality of therapeutic ultrasound transducers can as great as about 50 watts. In other embodiments, the output power for each of the plurality of therapeutic ultrasound transducers is between about 6 to about 10 watts.

In the illustrated embodiment of FIG. 1B, sides 130 and 150 vary in dimension along the Z direction, having dimension 134 at the attachment of sides 140 and 160, and dimension 136 at mid point 138. In certain embodiments, dimension 134 is between about 2 cm and about 4 cm. In certain embodiments, dimension 136 is between about 3 cm and about 8 cm. In other embodiments, Applicants' hand-held ultrasound emitting device comprises a parallelepiped, i.e. dimension 132 is substantially equal to dimension 134.

Referring to FIG. 1C, in certain embodiments Applicants' hand-held ultrasound emitting device 100 comprises housing 170 which includes top 110 and sides 130, 140, 150, and 160. In certain embodiments, housing 170 is integrally formed from one or more metallic materials. In certain embodiments, housing 170 is integrally molded from one or more polymeric materials. In certain embodiments, housing 170 is formed from one or more full density polymeric materials. In certain embodiments, those polymeric materials include polyethylene, polypropylene, polycarbonate, polystyrene, polyvinylchloride, combinations thereof, and the like.

In certain embodiments, those polymeric materials comprise one or more partial-density materials, i.e. one or more cellular materials. In certain embodiments, such cellular materials comprise one or more structural foam materials formed from the group which includes one or more polyurethanes, one or more polystyrenes, and combinations thereof, and the like.

Bottom 120 in combination with housing 170 comprises an enclosure. Bottom 120 includes interior surface 122 and exterior surface 124. In certain embodiments, bottom 120 is formed from metal, one or more polymeric materials, and combinations thereof. In certain embodiments, housing 170 is formed from one or more first polymeric materials and bottom 120 is formed from one or more second polymeric materials, where the one or more first polymeric materials differ from the one or more second polymeric materials.

In certain embodiments, bottom 120 is attached to housing 170 using adhesive bonding. In certain embodiments, bottom 120 is attached to housing 170 using conventional attachment means such as, for example, screws, nuts/bolts, rivets, and the like. In certain embodiments, bottom 120 can be releaseably affixed to housing 170, such that housing 170 can be used with a variety of differing sound head matrix assemblies, as described below.

A plurality of piezoelectric transducers are disposed on, or through, the exterior surface of the bottom portion of Applicants' device. Each piezoelectric transducer, sometimes referred to as a “sound head,” includes one or more piezoelectric materials. When an alternating current is applied to such a piezoelectric material, deformation occurs wherein the peizoelectric material expands and contracts. Such expansion and contraction crystal produces vibrations, i.e. sound waves.

In certain embodiments, Applicants' piezoelectric transducers comprise one or more ceramic materials having pronounced piezoelectric characteristics. In certain embodiments, Applicants' piezoelectric transducers comprise lead zirconate titanate (“PZT”). In other embodiments, Applicants' piezoelectric material comprises lead-magnesium-niobate lead titanate, hereafter referred to for brevity by the acronym PMN-PT. Such PMN-PT materials are described in U.S. Pat. No. 6,737,789.

In certain embodiments, Applicants' piezoelectric materials are formed from a thick-film ink, wherein one or more PZT and/or PMN-PT pastes are mixed with a powdered glass and an organic carrier, which is then printed onto the bottom portion of Applicants' device.

In certain embodiments, the plurality of piezoelectric transducers disposed on the exterior of Applicants' device comprise therapeutic ultrasound transducers. By “therapeutic ultrasound transducer,” Applicants mean a device that is capable of operating at between a 0.1 percent and a 100 percent duty cycle, and that emits therapeutic ultrasound energy. By “therapeutic ultrasound energy,” Applicants mean sound waves having a frequency between about 150 kilohertz and about 10 megahertz or higher, and a power level between about 0.1 watt/cm² and about 30 watts/cm². In certain embodiments, when operated continuously, the output power for each of the plurality of therapeutic ultrasound transducers can as great as about 50 watts. In other embodiments, the output power for each of the plurality of therapeutic ultrasound transducers is between about 6 to about 10 watts.

The plurality of therapeutic ultrasound transducers disposed on Applicants' device comprise a sound head matrix. In certain embodiments, Applicants' sound head matrix comprises a plurality of therapeutic ultrasound transducers are arranged in columns and rows.

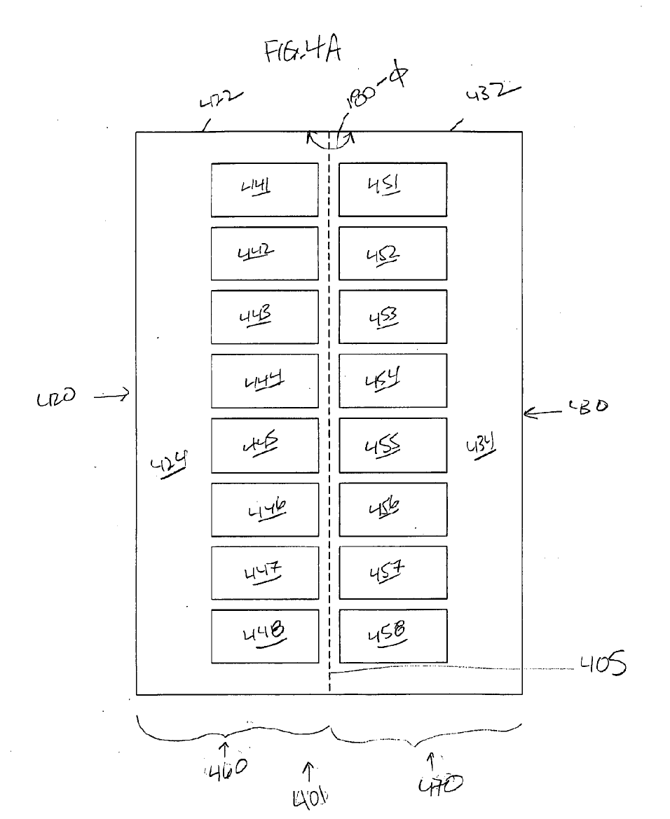

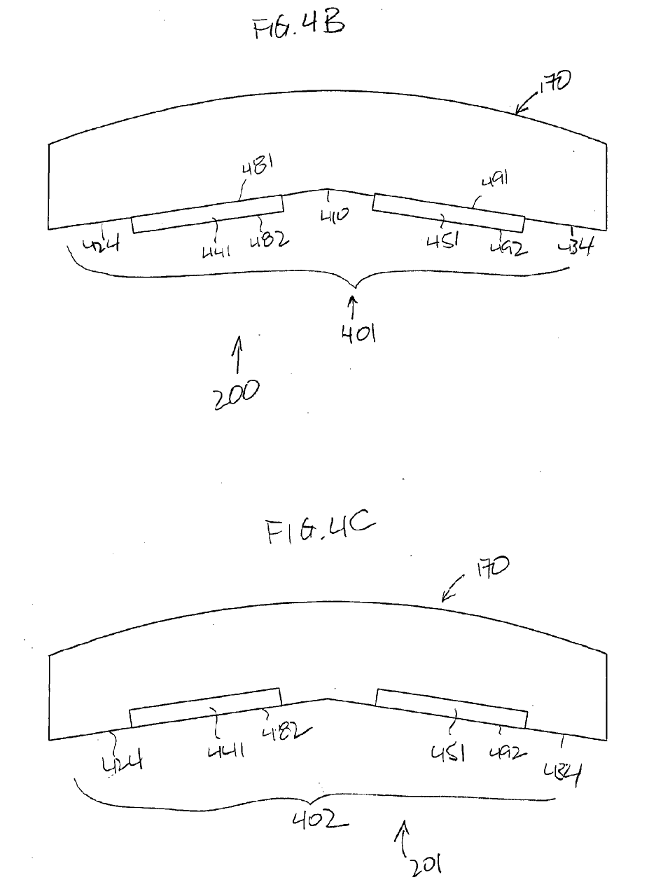

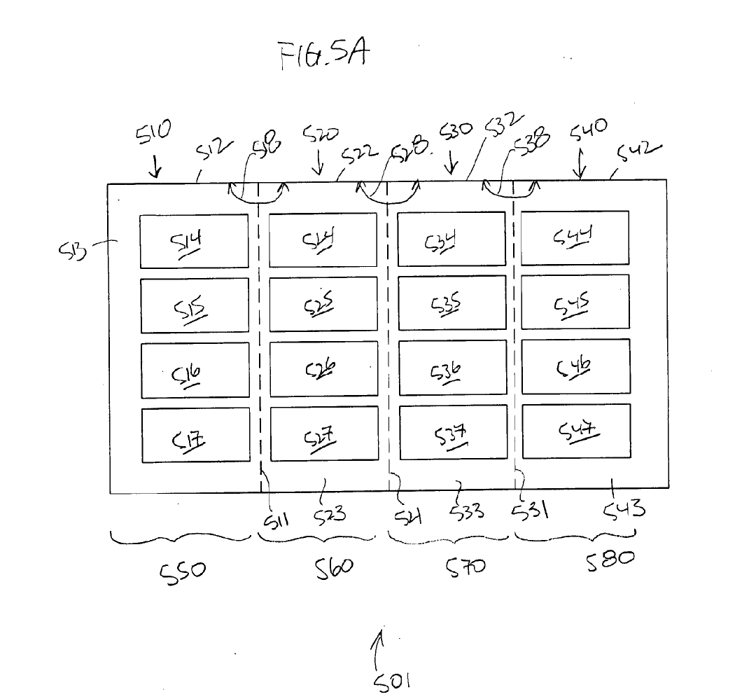

FIG. 4A shows one embodiment of Applicants' sound head matrix. In the illustrated embodiment of FIG. 4A, the sound head matrix comprises sixteen (16) therapeutic ultrasound transducers arranged in two columns of eight (8) transducers. Thus, sound head matrix of FIG. 4A comprises an 8×2 sound head matrix.

Each transducer comprising the sound head matrix of FIG. 4A is disposed on, or through, one of two planar members, either planar member 420 or planar member 430. In certain embodiments, planar member 420 and/or planar member 430 comprises a circuit substrate, wherein one or more electrical circuit components are attached to and/or through that circuit substrate. In certain embodiments, such a circuit substrate comprises what is sometimes referred to as a printed circuit board (“PCB”). In certain embodiments, planar member 420 and/or planar member 430 comprises a single-sided PCB. In certain embodiments, planar member 420 and/or planar member 430 comprises a double-sided PCB. In certain embodiments, planar member 420 and/or planar member 430 comprises a multilayer PCB. In certain embodiments, planar member 420 and/or planar member 430 comprises a metal core, i.e. copper for example, encapsulated with a ceramic coating.

In certain embodiments, planar member 420 and/or planar member 430 comprise a ceramic material. In certain embodiments, planar member 420 and/or planar member 430 comprise aluminum oxide. In certain embodiments, planar member 420 and/or planar member 430 comprise beryllium oxide.

In embodiments wherein housing 170 comprises one or more metallic components, and wherein planar members 420 and/or 430 comprise a ceramic material and/or a ceramic material encapsulating a copper core, planar members 420 and/or 430 conduct heat generated by the plurality of ultrasound emitters from the core of Applicants' device to the metallic housing, i.e. the circuit substrates in combination with the housing, comprise, inter alia, an integrated heat sink assembly which continuously dissipates heat from Applicants' hand-held device to the environment.

Planar member 420 is continuously attached to planar member 430 at common edge 405. Transducers 441, 442, 443, 444, 445, 446, 447, and 448, are disposed on, or through, surface 424 of planar member 420. Transducers 441, 442, 443, 444, 445, 446, 447, and 448, in combination with planar member 420, comprises planar assembly 460. Transducers 451, 452, 453, 454, 455, 456, 457, and 458, are disposed on, or through, surface 434 of planar member 430. Transducers 451, 452, 453, 454, 455, 456, 457, and 458, in combination with planar member 430, comprises planar assembly 470.

Planar assembly 460 in combination with planar assembly 470 comprises sound head matrix assembly 401. In certain embodiments, sound head matrix assembly 401 comprises a substantially flat structure. In other embodiments, sound head matrix assembly 401 is not flat, i.e. the dihedral angles formed by the intersection of assemblies 460 and 470 do not equal 180 degrees.

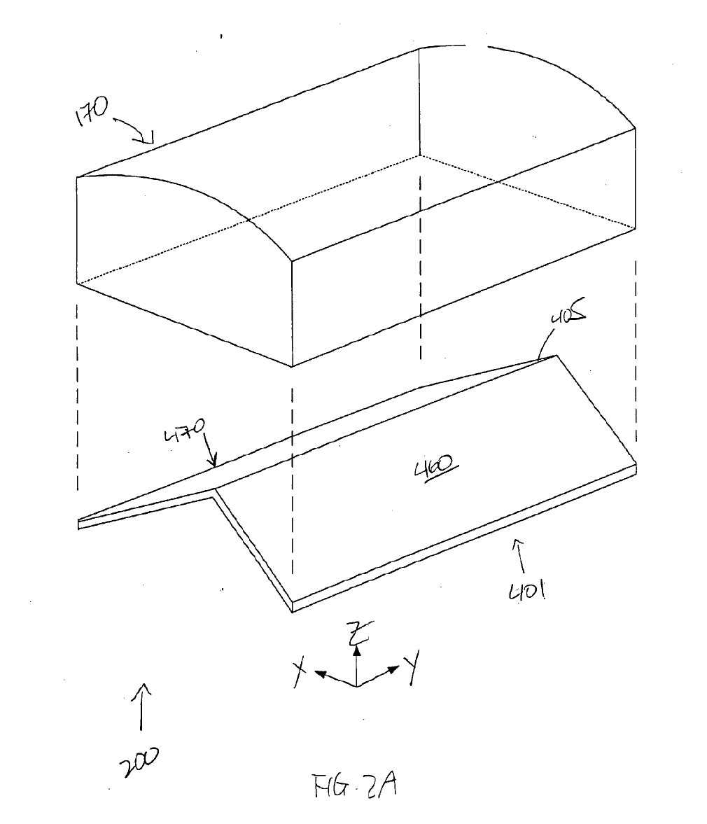

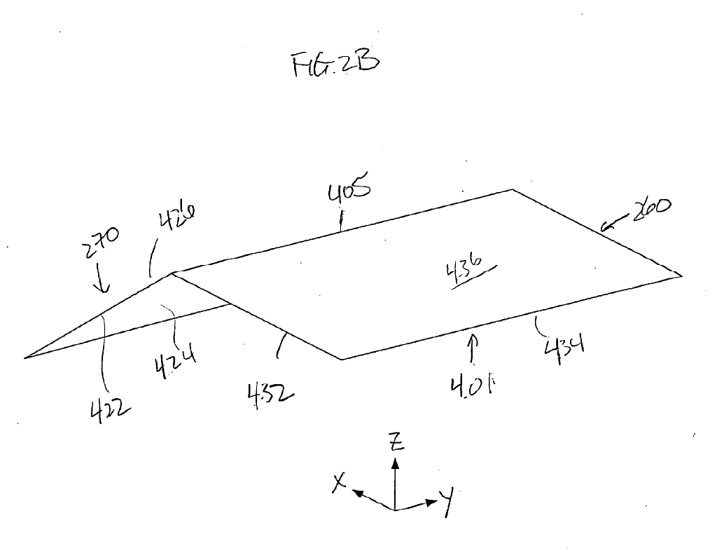

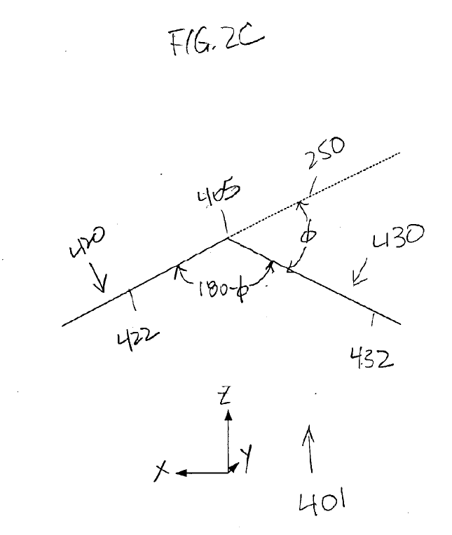

Referring to FIG. 2A, device 200 includes housing 170 (FIG. 1C) in combination with an “offset” embodiment of sound head matrix assembly 401. As described above, sound head matrix assembly 401 includes planar assembly 460 in combination with planar assembly 470, where planar assembly 460 is continuously joined to planar assembly 470 along common edge 405. Planar assembly 460 lies in a first plane, and planar assembly 470 lies in a second plane. That first plane intersects the second plane along common edge 405 to form an interior dihedral angle, as defined herein, less than 180 degrees.

Referring now to FIGS. 2A and 2B, planar assembly 460 includes edge 422. Planar assembly 470 includes edge 432. Edge 422 meets edge 432 at seam 405. Dotted line 250 represents the extension of edge 422 past seam 405. As shown in FIG. 2C, angle Φ represents the angle formed between edge 432 and extension line 250. For purposes of this Application, planar assembly 460 is “offset” from planar assembly 470 by angle Φ. As those skilled in the art will. appreciate, the interior dihedral angle, in degrees, formed by the intersection of planar assembly 460 and planar assembly 470 is 180−Φ.

In certain embodiments, angle Φ is between about 5 degrees and about 25 degrees. In certain embodiments, angle Φ is between about 10 degrees and about 20 degrees. In certain embodiments, angle Φ is about 13 degrees.

As those skilled in the art will appreciate, the interior dihedral angle formed by planar assembly 460 and planar assembly 470 is inversely proportional to the offset angle Φ. Therefore, as Φ increases from 0 degrees, the dihedral angle decreases from 180 degrees. Thus, where planar assembly 460 is “offset” from planar assembly 470 by, for example, 15 degrees, then the interior dihedral angle formed by planar assembly 460 and planar assembly 470 is 165 degrees.

FIG. 4B shows a side view of apparatus 200 which includes housing 170 in combination with an offset sound head matrix assembly 401. Transducer 441 comprises a first side 481 and an opposing second side 482. Transducer 451 includes a first side 491 and an opposing second side 492. In the illustrated embodiment of FIG. 4B, side 481 of transducer 441 is disposed on surface 424 of planar member 420, and side 491 of transducer 451 is disposed on surface 434 of planar member 430. As those skilled in the art will appreciate, transducers 441 may include one or more leads which extend through holes, i.e. vias, drilled through planar member 420. In other embodiments, transducer 441 comprises what is sometimes called a “surface mounted” device, wherein that surface mounted device is attached to a solder pad disposed on surface 424.

FIG. 4C shows a side view of apparatus 201 which includes housing 170 in combination with an offset sound head matrix assembly 402. Sound head matrix assembly 402 is identical to sound head matrix assembly 401 except that each of the plurality of therapeutic ultrasound transducers extends through a planar member rather than being disposed on that planar member. For example in the illustrated embodiment of FIG. 4C, transducer 441 is disposed through planar member 420 such that surface 482 of transducer 441 is flush with surface 424 of planar assembly 460. Similarly in this embodiment, transducer 451 is disposed through planar member 430 such that surface 492 of transducer 451 is flush with surface 434 of planar assembly 470.

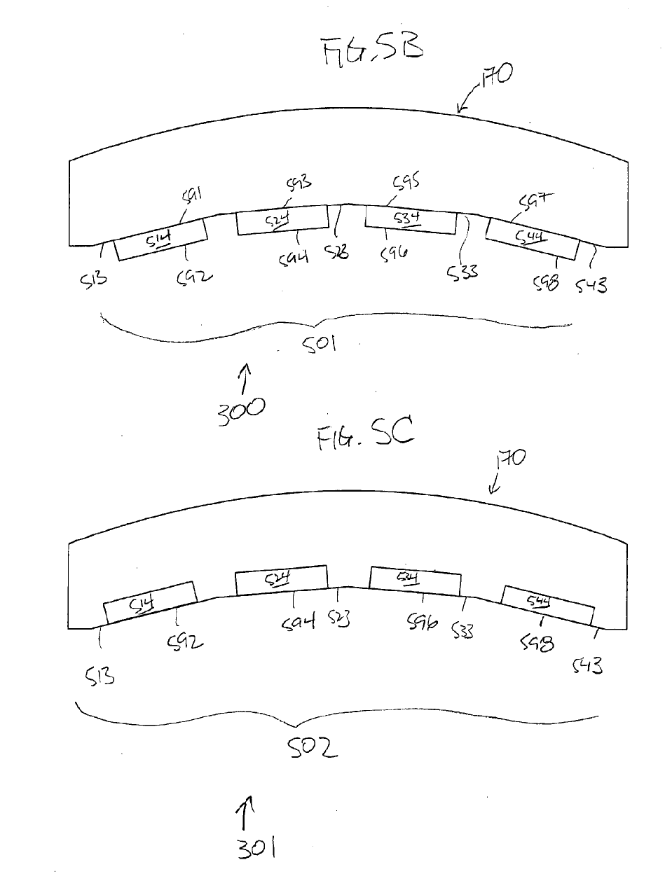

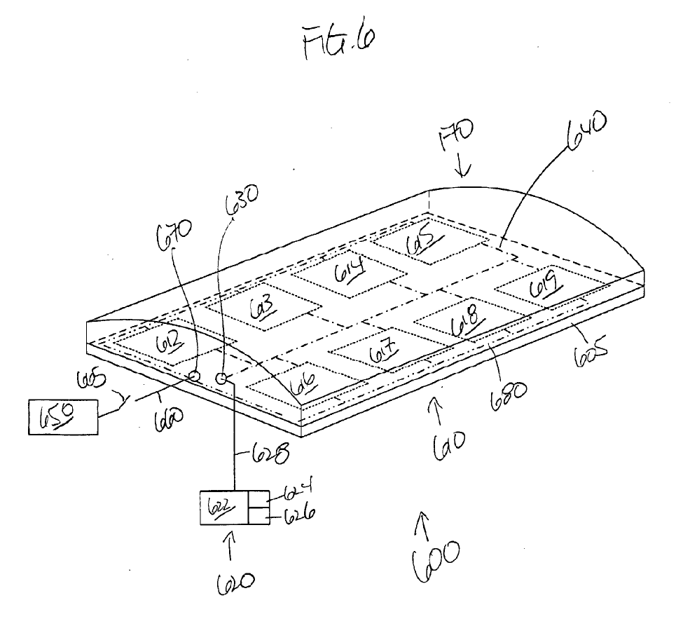

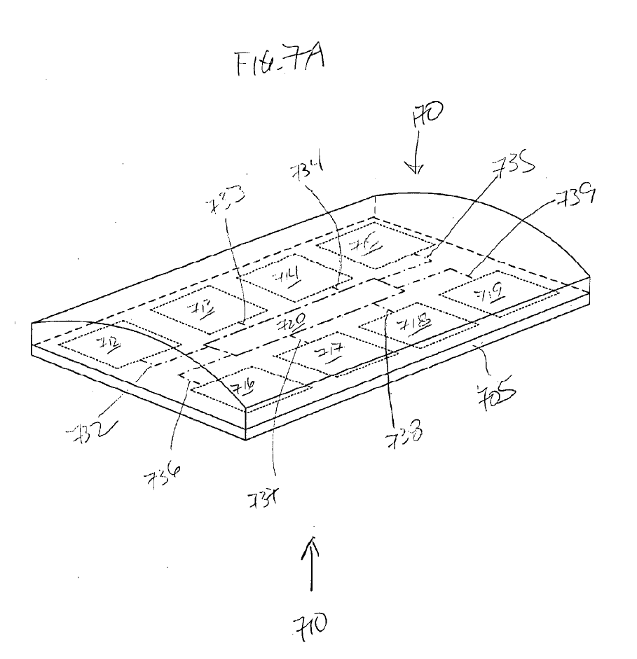

FIG. 5A shows another embodiment of Applicants' sound head matrix. In the illustrated embodiment of FIG. 5A, the sound head matrix comprises sixteen (16) therapeutic ultrasound transducers arranged in four columns of four transducers. Thus, sound head matrix of FIG. 5A comprises an 4×4 sound head matrix.

Each transducer comprising the sound head matrix of FIG. 5A is disposed on, or through, one of four planar members, namely planar member 510, or planar member 520, or planar member 530, or planar member 540. Planar member 510 is continuously attached to planar member 520 at common edge 511. Transducers 514, 515, 516, and 517, are disposed on, or through, surface 513 of planar member 510. Transducers 514, 515, 516, and 517, in combination with planar member 510, comprise planar assembly 550. Angle 518 comprises the interior dihedral angle formed by the intersection of planar member 510 with planar member 520.

In certain embodiments, angle 518 is about 180 degrees. In these embodiments, planar member 510 is not offset from planar member 520, i.e. planar member 510 in combination with planar member 520 comprises a substantially flat assembly. In other embodiments, angle 518 is less than 180 degrees, i.e. planar member 510 is offset from planar member 520.

In certain embodiments, planar members 510 and 520 are integrally formed to include angle 518. In other embodiments, planar members 510 and 520 are individually formed, and subsequently attached using conventional attachment methods.

Planar member 520 is continuously attached to planar member 530 at common edge 521. Transducers 524, 525, 526, and 527, are disposed on, or through, surface 523 of planar member 520. Transducers 524, 525, 526, and 527, in combination with planar member 520, comprise planar assembly 560. Angle 528 comprises the interior dihedral angle formed by the intersection of planar member 520 with planar member 530.

In certain embodiments, angle 528 is about 180 degrees. In these embodiments, planar member 520 is not offset from planar member 530, i.e. planar member 520 in combination with planar member 530 comprises a substantially flat assembly. In other embodiments, angle 528 is less than 180 degrees, i.e. planar member 520 is offset from planar member 530.

In certain embodiments, planar members 520 and 530 are integrally formed to include angle 528. In other embodiments, planar members 520 and 530 are individually formed, and subsequently attached using conventional attachment methods.

Planar member 530 is continuously attached to planar member 540 at common edge 531. Transducers 534, 535, 536, and 537, are disposed on, or through, surface 533 of planar member 530. Transducers 534, 535, 536, and 537, in combination with planar member 530, comprise planar assembly 570. Angle 538 comprises the interior dihedral angle formed by the intersection of planar member 530 with planar member 540.

In certain embodiments, angle 538 is about 180 degrees. In these embodiments, planar member 530 is not offset from planar member 540, i.e. planar member 530 in combination with planar member 540 comprises a substantially flat assembly. In other embodiments, angle 538 is less than 180 degrees, i.e. planar member 530 is offset from planar member 540.

In certain embodiments, planar members 530 and 540 are integrally formed to include angle 538. In other embodiments, planar members 530 and 540 are individually formed, and subsequently attached using conventional attachment methods.

Transducers 544, 545, 546, and 547, are disposed on, or through, surface 543 of planar member 530. Transducers 544, 545, 546, and 547, in combination with planar member 540, comprise planar assembly 580.

Planar assemblies 550, 560, 570, and 580, in combination, comprise sound head matrix assembly 501. In certain embodiments, sound head matrix assembly 501 comprises a substantially flat structure. In other embodiments, sound head matrix assembly 501 is not flat.

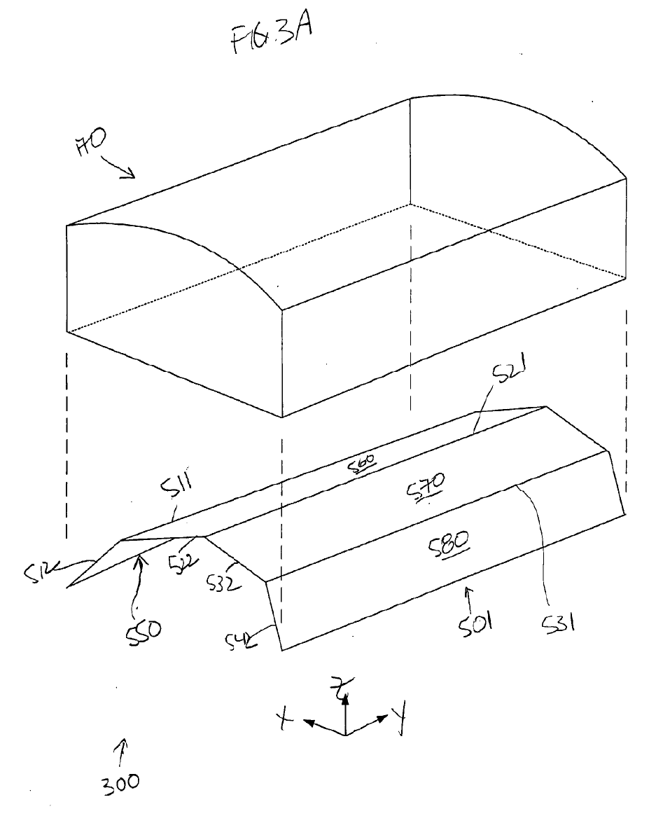

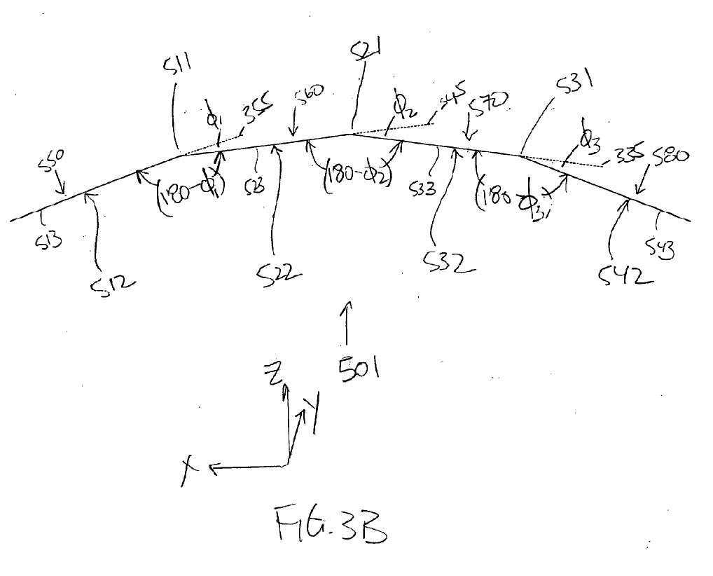

Referring to FIGS. 3A and 3B, device 300 includes housing 170 (FIG. 1C) in combination with sound head matrix assembly 501. Edge 512 of planar assembly 550 meets edge 522 of planar assembly 560 at seam 511. Dotted line 355 represents the extension of edge 512 past seam 511. As shown in FIG. 3B, angle Φ represents the angle formed between edge 522 and extension line 335. For purposes of this Application, planar assembly 550 is “offset” from planar assembly 560, where the offset angle is angle Φ1. As those skilled in the art will appreciate, the interior dihedral angle, in degrees, formed by the intersection of planar assembly 550 and planar assembly 560 is 180−Φ1. By “interior dihedral angle,” Applicants' mean the angle formed between surface 513 and surface 523.

In certain embodiments, angle Φ1 is between about 5 degrees and about 25 degrees. In certain embodiments, angle Φ1 is between about 8 degrees and about 15 degrees. In certain embodiments, angle Φ1 is about 13 degrees.

Edge 522 of planar assembly 560 meets edge 532 of planar assembly 570 at seam 521. Dotted line 345 represents the extension of edge 522 past seam 521. As shown in FIG. 3B, angle Φ2 represents the angle formed between edge 532 and extension line 345. For purposes of this Application, planar assembly 560 is “offset” from planar assembly 570, where the offset angle is angle Φ2. As those skilled in the art will appreciate, the interior dihedral angle, in degrees, formed by the intersection of planar assembly 560 and planar assembly 570 is 180−Φ1. By “interior dihedral angle,” Applicants' mean the angle formed between surface 523 and surface 533.

In certain embodiments, angle Φ2 is between about 5 degrees and about 25 degrees. In certain embodiments, angle Φ2 is between about 8 degrees and about 15 degrees. In certain embodiments, angle Φ2 is about 10 degrees.

Edge 532 of planar assembly 570 meets edge 542 of planar assembly 570 at seam 531. Dotted line 335 represents the extension of edge 532 past seam 531. As shown in FIG. 3B, angle Φ3 represents the angle formed between edge 542 and extension line 335. For purposes of this Application, planar assembly 570 is “offset” from planar assembly 580, where the offset angle is angle Φ3. As those skilled in the art will appreciate, the interior dihedral angle, in degrees, formed by the intersection of planar assembly 570 and planar assembly 580 is 180−Φ1. By “interior dihedral angle,” Applicants' mean the angle formed between surface 533 and surface 543.

In certain embodiments, angle Φ3 is between about 5 degrees and about 25 degrees. In certain embodiments, angle Φ3 is between about 8 degrees and about 15 degrees. In certain embodiments, angle Φ3 is about 13 degrees.

In certain embodiments, two or more of offset angles Φ1, Φ2, and/or Φ3, are substantially the same. By “substantially the same,” Applicants means within about plus or minus ten percent or less. In other embodiments, two or more of offset angles Φ1, Φ2, and/or Φ3, differ.

FIG. 5B shows a side view of apparatus 300 which includes housing 170 in combination with a multiply offset sound head matrix assembly 501. Transducers 514, 524, 534, and 544, each comprise a first side 591, 593, 595, and 597, respectively, and an opposing second side 592, 594, 596, and 598, respectively.

In the illustrated embodiment of FIG. 5B, side 591 of transducer 441, and side 593 of transducer 524, and side 595 of transducer 534, and side 597 of transducer 544, respectively, are disposed on surface 513 of planar assembly 550, surface 523 of planar assembly 560, surface 533 of planar assembly 570, and surface 543 of planar assembly 580, respectively. Transducers 515, 516, 517, 525, 526, 527, 535, 536, 537, 545, 546, and 547, are similarly attached to their respective planar assemblies.

As those skilled in the art will appreciate, the plurality of transducers comprising sound head matrix assembly 501 may include one or more leads which extend through holes, i.e. vias, drilled through one of the four planar assemblies. In other embodiments, the plurality of transducers comprising sound head matrix 501 each comprise what is sometimes called a “surface mounted” device, wherein that surface mounted device is attached to a solder pad disposed on surface 513, or surface 523, or surface 533, or surface 443.

FIG. 5C shows a side view of apparatus 301 which includes housing 170 in combination with an offset sound head matrix assembly 502. Sound head matrix assembly 502 is identical to sound head matrix assembly 501 except that each of the plurality of therapeutic ultrasound transducers extends through a planar assembly rather than being disposed on the exterior surface of that planar assembly. For example in the illustrated embodiment of FIG. 5C, transducers 514, 524, 534, and 544, respectively, are disposed through planar assembly 550, planar assembly 560, planar assembly 570, and planar assembly 580, respectively, such that surface 592 of transducer 514 is flush with surface 513 of planar assembly 550, and, such that surface 594 of transducer 524 is flush with surface 523 of planar assembly 560, and such that surface 596 of transducer 534 is flush with surface 533 of planar assembly 570, and such that surface 598 of transducer 544 is flush with surface 543 of planar assembly 580.

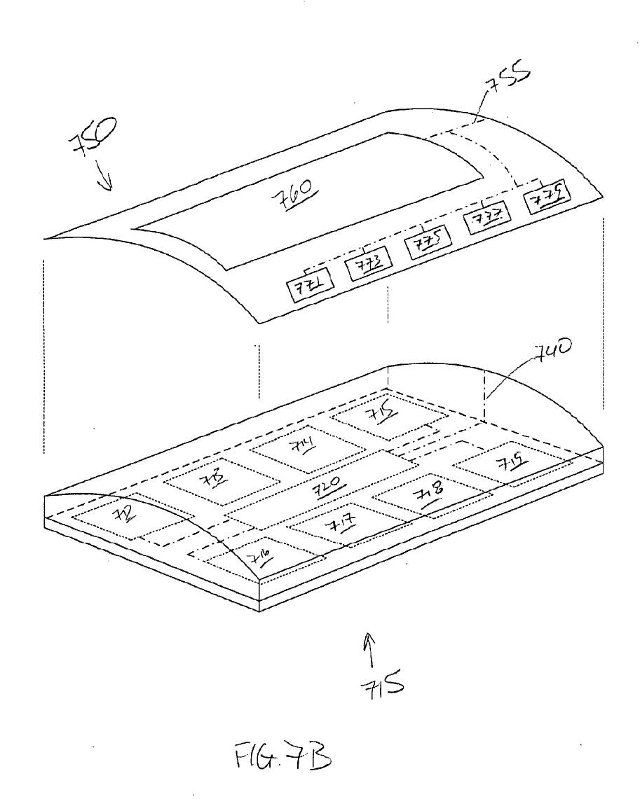

FIG. 6 shows one embodiment of Applicants' therapeutic ultrasound apparatus 600. Apparatus 600 includes hand-held ultrasonic device 610, external controller 620, and power source 650. Power source 650 provides power to device 610 by power cable 660. In certain embodiments, Applicants' system 600 includes power switch 665. In the illustrated embodiment of FIG. 6 power switch 665 is disposed in power cable 660. In other embodiments, switch 665 is disposed on power source 650. In other embodiments, switch 665 is disposed on the outer surface of device 610. Power switch 665 can comprise any suitable power switching device, and may take the form of, for example, a rocker switch, a toggle switch, a push to operate switch, and the like.

Device 610 includes housing 170 and sound head matrix assembly 605. In the illustrated embodiment of FIG. 6, Applicants' sound head matrix assembly 605 comprises a 4×2 sound head matrix. As a general matter, Applicants' sound head matrix assembly comprises a Y×Z sound head matrix, wherein Y represents the number of transducers in a column, and wherein Z represents the number of columns, wherein Y is greater than or equal to 1, and less than or equal to about 10, and wherein Z is greater than or equal to 1 and less than or equal to about 6.

For example in certain embodiments, Applicants' hand-held ultrasonic device 610 comprises an 8×2 sound head matrix, such as the sound head matrix recited in FIG. 4A. In certain embodiments, Applicants' hand-held ultrasonic device 610 comprises a 4∴4 sound head matrix, such as the sound head matrix recited in FIG. 5A.

In the illustrated embodiment of FIG. 6, Applicants' sound head matrix assembly is substantially flat. In other embodiments, Applicants' sound head matrix assembly comprises (N) offset planar assemblies, wherein (N) is greater than or equal to 2 and less than or equal to about 6.

For example, in certain embodiments, Applicants' hand-held ultrasonic device 610 comprises offset sound head matrix assembly 401 (FIGS. 2A, 3A, 4A, 4B) ), where that sound head matrix assembly comprises a Y×2 sound head matrix. In other embodiments, Applicants' hand-held ultrasonic device 610 comprises offset sound head matrix assembly 402 (FIG. 4C), where that sound head matrix assembly comprises a Y×2 sound head matrix. In other embodiments, Applicants' hand-held ultrasonic device 610 comprises offset sound head matrix assembly 501 (FIGS. 5A, 5B), where that sound head matrix assembly comprises a Y×4 sound head matrix. In other embodiments, Applicants' hand-held ultrasonic device 610 comprises offset sound head matrix assembly 502 (FIG. 5C), where that sound head matrix assembly comprises a Y×4 sound head matrix.

Controller 620 is interconnected with hand-held device 610 by communication link 628. In certain embodiments, communication link 628 is selected from the group which includes a serial interconnection, such as RS-232 or RS-422, an ethernet interconnection, a SCSI interconnection, a Fibre Channel interconnection, an ESCON interconnection, a FICON interconnection, a Local Area Network (LAN), a private Wide Area Network (WAN), a public wide area network, Storage Area Network (SAN), Transmission Control Protocol/Internet Protocol (TCP/IP), the Internet, and combinations thereof.

Communication link 628 can be releaseably attached to coupling 630 disposed on housing 170. Coupling 630 is interconnected with control bus 640. Control bus 640 is interconnected to each transducer comprising Applicants' sound head matrix assembly 610.

In certain embodiments, controller 620 provides control signals to hand-held device 610 wirelessly. In these wireless embodiments, communication link 628 comprises a first antenna coupled to controller 620 and coupling 630 comprises a second antenna coupled to communication bus 640.

Controller 620 includes processor 622, memory 624, and device microcode 626. In certain embodiments, memory 624 comprises one or more nonvolatile memory devices. In certain embodiments, such nonvolatile memory is selected from the group which includes one or more EEPROMs (Electrically Erasable Programmable Read Only Memory), one or more flash PROMs (Programmable Read Only Memory), battery backup RAM, hard disk drive, combinations thereof, and the like.

In certain embodiments, microcode 626 is stored in memory 624. Device microcode 626 comprises instructions residing in memory, such as for example memory 624, where those instructions are executed by processor 622 to implement the selected operational mode for the plurality of transducers comprising Applicants' sound head matrix assembly.

In certain embodiments, device microcode 626 comprises instructions residing in memory, such as for example memory 624, where those instructions are executed by processor 622 to cause each of the plurality of therapeutic ultrasound transducers comprising Applicants' sound head matrix assembly 605 to operate continuously. In other embodiments, device microcode 626 comprises instructions residing in memory, such as for example memory 624, where those instructions are executed by processor 622 to cause each of the plurality of therapeutic ultrasound transducers comprising Applicants' sound head matrix assembly 605 to operate discontinuously.

As a general matter, such discontinuous operation modes include embodiments wherein each of the plurality of therapeutic ultrasound transducers comprising Applicants' sound head matrix assembly 605 operates on a duty cycle from about 0.1 percent to 100 percent. In certain embodiments, such discontinuous operation modes include embodiments wherein each of the plurality of therapeutic ultrasound transducers comprising Applicants' sound head matrix assembly 605 operates on a duty cycle selected from the group comprising a 20 percent duty cycle, a 40 percent duty cycle, a 60 percent duty cycle, and an 80 percent duty cycle.

In certain of these discontinuous operational modes, each of the plurality of therapeutic ultrasound transducers comprising Applicants' sound head matrix assembly 605 operates independently of any of the other transducer, i.e. each transducer is alternately turned on and off randomly. In other embodiments, an entire column of transducers operates at the same time, while transducers comprising other columns do not operate. In other embodiments, an entire row of transducers operates at the same time, while transducers comprising other rows do not operate.

The following examples are presented to further illustrate to persons skilled in the art how to make and use Applicants' invention, and to identify a presently preferred embodiment thereof. These examples are not intended as limitations, however, upon the scope of the invention.

### EXAMPLE I

For example and referring to FIG. 5A, in certain embodiments a first column of therapeutic ultrasound transducers, which includes transducers 514, 515, 516, and 517, emit therapeutic ultrasound energy while a second column which includes transducers 524, 525, 526, 527, and while a third column which includes transducers 534, 535, 536, 537, and while a fourth column which includes transducers 544, 545, 546, and 547, do not emit therapeutic ultrasound energy. Thereafter, the transducers comprising the second column emit energy while the transducers in the first, third, and fourth columns do not. Applicants' method includes embodiments wherein any pattern of sequential activation of columns of therapeutic ultrasound transducers.

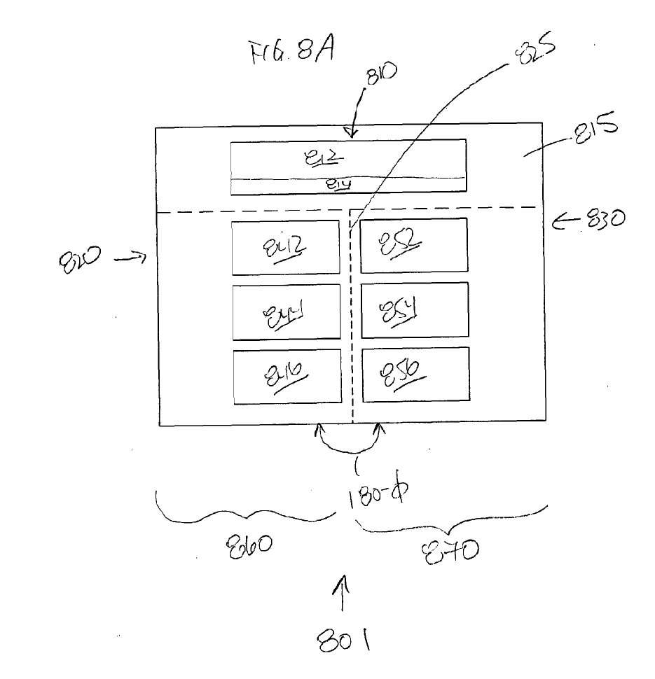

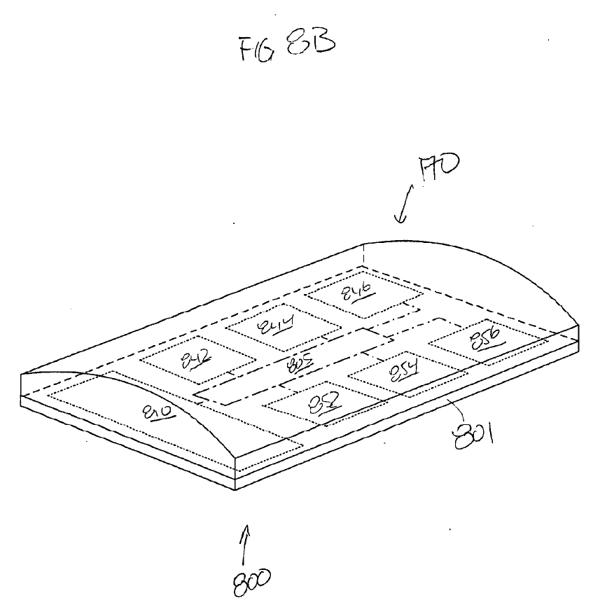

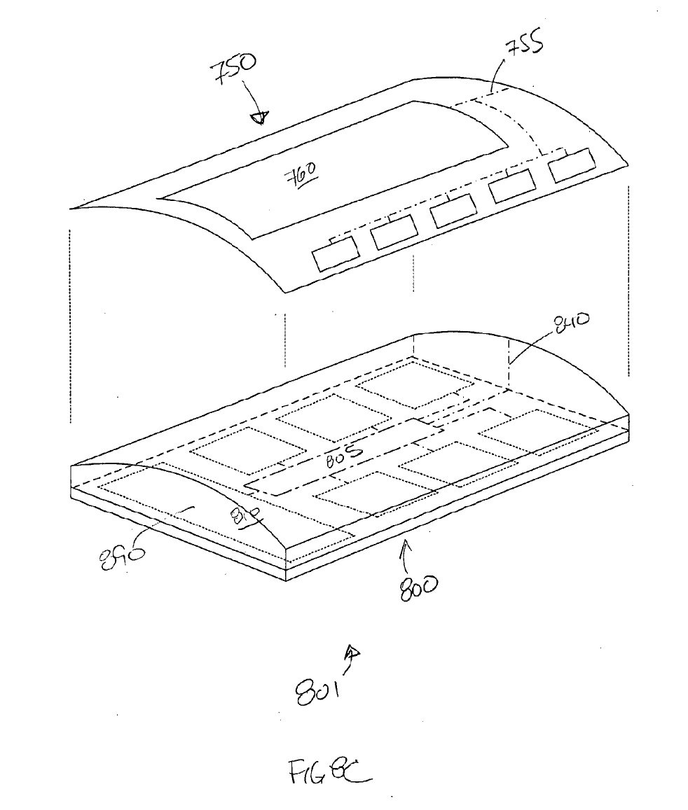

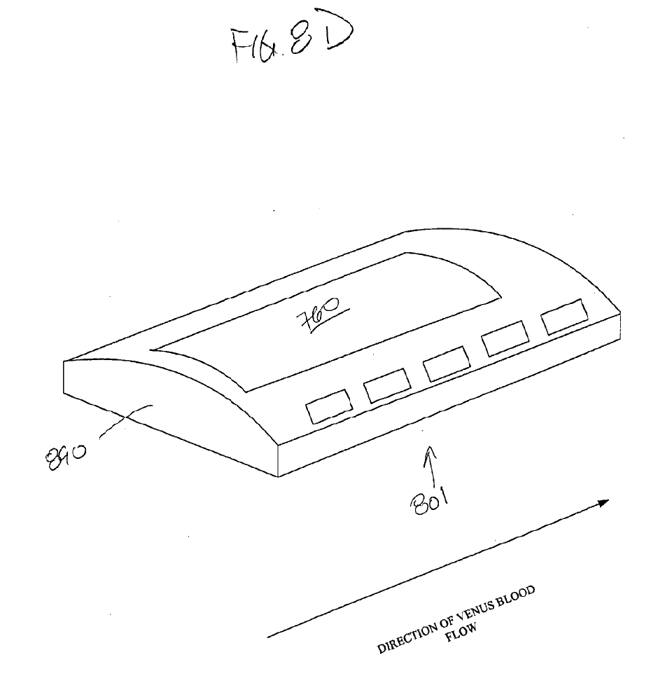

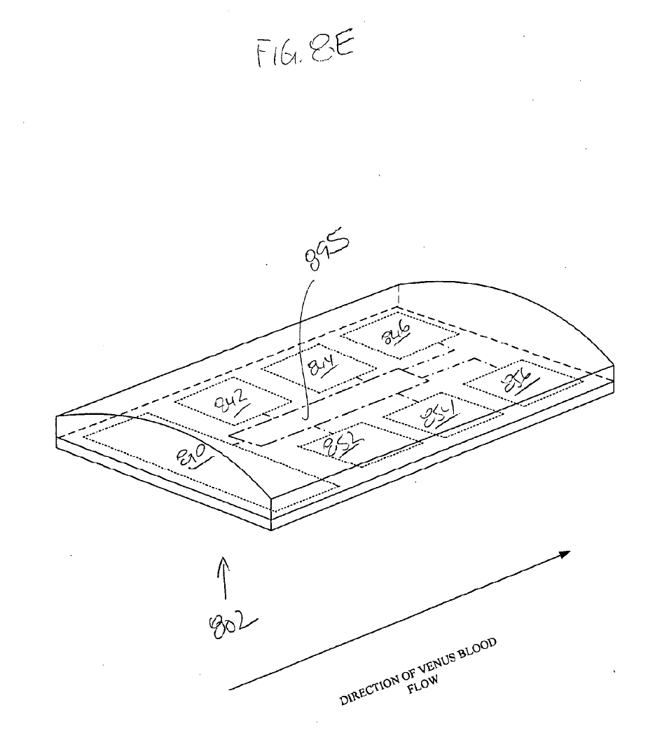

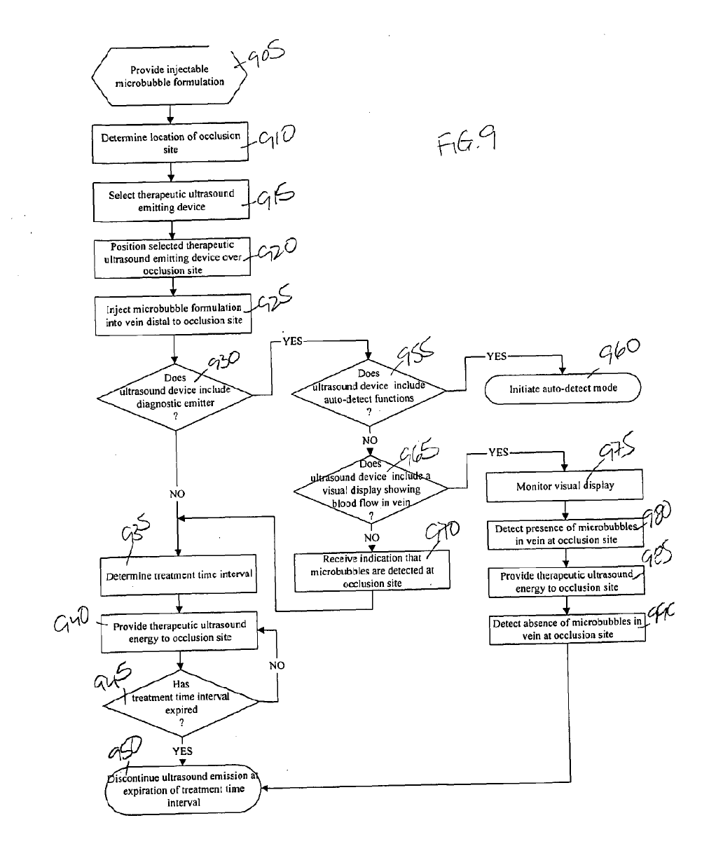

As a further example, in embodiments wherein Applicants' sound head matrix comprises two or more columns, controller 620 (FIG. 6)/720 (FIGS. 7A, 7B)/805 (FIGS. 8A, 8B), causes the ultrasound transducers arranged in a first column of that sound head matrix to emit ultrasound energy during a first time interval, and causes the ultrasound transducers in a second column of that sound head matrix to emit ultrasound energy during a second time interval, where the first time interval differs from the second time interval. Applicants' method may define a treatment duration, and controller 620 (FIG. 6)/720 (FIGS. 7A, 7B)/805 (FIGS. 8B, 8C), 895 (FIG. 8E), retrieves that pre-determined treatment duration, and alternates the first time interval and the second time interval throughout that treatment duration.

### EXAMPLE II

In another example, a first row of therapeutic ultrasound transducers, which includes transducers 514, 524, 534, and 544, emit therapeutic ultrasound energy while a second row which includes transducers 515, 525, 535, 534, and while a third row which includes transducers 516, 526, 536, 545, and while a fourth row which includes transducers 517, 527, 537, and 547, do not emit therapeutic ultrasound energy. Thereafter, the transducers comprising the second row emit energy while the transducers in the first, third, and fourth rows do not. Applicants' method includes embodiments wherein any pattern of sequential activation of rows of therapeutic ultrasound transducers.

As a further example, in embodiments wherein Applicants' sound head matrix comprises two or more rows, controller 620 (FIG. 6)/720 (FIGS. 7A, 7B)/805 (FIGS. 8B, 8C), 895 (FIG. 8E), causes the ultrasound transducers arranged in a first row of that sound head matrix to emit ultrasound energy during a first time interval, and causes the ultrasound transducers in a second row of that sound head matrix to emit ultrasound energy during a second time interval, where the first time interval differs from the second time interval. Applicants' method may define a treatment duration, and controller 620 (FIG. 6)/720 (FIGS. 7A, 7B)/805 (FIGS. 8B, 8C), 895 (FIG. 8E), retrieves that pre-determined treatment duration, and alternates the first time interval and the second time interval throughout that treatment duration.

In certain embodiments, controller 620 comprises a computer, which in addition to memory 624 and microcode 624, further includes one or more input devices, such as for example a key board, a mouse, a pointing device, and the like. In certain embodiments, that computer further includes one or more output devices, such as for example one or more monitors, one or more printers, and the like.

In certain embodiments of Applicants' apparatus, the external control circuitry of FIG. 6, i.e. controller 620, is disposed within Applicants' hand-held ultrasonic device. Referring to FIG. 7A, hand-held device 710 includes the elements of device 610 in combination with controller 720. For clarity of illustration, FIG. 7 does not include power source 650, power cable 660, or power bus 605. Controller 720 comprises processor 622, memory 624, and microcode 626.

Applicants' hand-held ultrasonic device 710 includes controller 720 which is interconnected to each of a plurality of therapeutic ultrasound transducers 712, 713, 714, 715, 716, 717, 718, and 719, via communication links 732, 733, 734, 735, 736, 737, 738, and 739, respectively.

For further clarity of illustration, the illustrated embodiment of FIG. 7A includes 4×2 sound head matrix assembly 705. As a general matter, sound head matrix assembly 705 comprises a Y×Z sound head matrix, where that Y×Z sound head matrix is described above, and where that Y×Z sound head matrix may comprise a substantially flat assembly, or that Y×Z sound head matrix assembly may comprise (N) offset planar assemblies. In certain embodiments, controller 720 comprises an application specific integrated circuit, i.e. an “ASIC,” which integrates the functions of processor 622, memory 624, and microcode 626.

Referring now to FIG. 7B, Applicants' hand-held ultrasonic device 715 includes the elements of device 710 (FIG. 7A) in combination with integrated information input/output (“I/O”) device 750. In the illustrated embodiment of FIG. 7B, I/O device 750 includes a visual display device 760 and a plurality of input device/touch screens 771, 773, 775, 777, and 779. In certain embodiments, visual display device 760 comprises an LCD device. I/O device communicates with controller 720 via communication links 740 and 755.

In certain embodiments, Applicants' hand-held ultrasonic device includes one or more diagnostic ultrasound emitters in combination with a plurality of therapeutic ultrasound emitters. Referring to FIG. 8A, sound head matrix assembly 801 includes diagnostic ultrasound transceiver 810, and a 2×3 sound head matrix comprising 6 therapeutic ultrasound emitters. Ultrasound transceiver 810 includes diagnostic ultrasound emitter 812 and receiving device 814. By “diagnostic ultrasound emitter,” Applicants' mean a device which is capable of emitting diagnostic ultrasound energy having a output power of between about 0.5 and about 1 milliwatt per cm² at a frequency of between about 7 and about 13 megahertz. Emitter 812 produces and emits ultrasound waves. Receiver 814 detects emissions reflected back to transceiver 810 by various underlying body tissues. Those reflected emissions are processed by the controller, such as for example controller 620 and/or controller 720, and/or controller 805, and that controller causes a visual display device, such as visual display device 760 to display an image of the tissue structure underlying the diagnostic ultrasound transceiver.

Any of the various types of diagnostic ultrasound imaging devices may be employed in the practice of the invention, the particular type or model of the device not being critical to the method of the invention. Also suitable are devices designed for administering ultrasonic hyperthermia, such devices being described in U.S. Pat. Nos. 4,620,546, 4,658,828, and 4,586,512, the disclosures of each of which are hereby incorporated herein by reference in their entirety. Preferably, the device employs a resonant frequency (RF) spectral analyzer.

Therapeutic ultrasound emitters 842, 844, and 846, are disposed on, or through, planar member 820. Emitters 842, 844, and 846, in combination with planar member 820, comprise planar assembly 860. Therapeutic ultrasound emitters 852, 854, 856, are disposed on, or through, planar member 830. Emitters 852, 854, and 856, in combination with planar member 830, comprise planar assembly 870.

Planar assembly 860 is continuously attached to planar assembly 870 at seam 825. In certain embodiments, the dihedral angle formed by the intersection of planar assembly 860 and planar assembly 870 is 180 degrees, i.e. the angle Φ shown in FIG. 8A is zero. In other embodiments, planar assembly 860 is offset from planar assembly 870, i.e. the angle Φ shown in FIG. 8A is greater than zero.

Referring now to FIG. 8B, Applicants' hand-held device 800 includes sound head matrix assembly 801 in combination with controller 805 and housing 170. Controller 805 includes a processor, such as processor 622, memory, such as memory 624, and device microcode, such as microcode 626, to operate the plurality of therapeutic emitters 842, 844, 846, 852, 854, and 856, and microcode to operate diagnostic transceiver 810.

In certain embodiments, Applicants' hand-held ultrasound device 800 includes an integral information input/output device. Referring now to FIG. 8C, device 801 includes hand-held device 800 in combination with integrated I/O device 750. Controller 805 communicates with I/O device 750 via communication links 804 and 755. Diagnostic transceiver 810 is internally disposed within device 801 adjacent end 890. In these embodiments, controller 805 includes a processor, such as processor 622, memory, such as memory 624, and device microcode, such as microcode 626, to operate the plurality of therapeutic emitters 842, 844, 846, 852, 854, and 856, and microcode to operate diagnostic transceiver 810, and microcode to operate visual display device 760.

Referring now to FIG. 8D, device 801 can be removeably affixed to, for example, a patient's leg in order to direct ultrasound energy into the tissues of that leg. In certain embodiments, Applicants' therapeutic method includes injecting microbubbles into a blood vessel distal to an occlusion in that vessel. Device 801 is positioned such that when the microbubbles approach the occasion site of the vessel, ultrasound energy produced by device 801 causes those bubbles to rupture, thereby removing all or part of the occlusion.

When using device 801, the diagnostic transceiver is first made operational. As those skilled in the art will appreciate, that diagnostic transceiver continuously emits relatively low power level ultrasound waves. The various body tissues differentially reflect a portion of those sound waves. The diagnostic transceiver detects those reflected signals. Controller 805 processes those reflected signals and generates an image signal. That image signal is provided to display device 760 which visually displays an image of the tissues and structures underlying device 801.

By monitoring display device 760, the medical provider can determine when the injected microbubbles have reached the occlusion site. At that time, the medical provider than causes the plurality of therapeutic ultrasound emitters to produce ultrasound energy having a higher power level than the diagnostic power levels emitted by transceiver 810. Those higher power ultrasound energy causes the microbubbles to rupture. After the flow of the injected microbubbles ceases, the medical provider then discontinues emission of the therapeutic ultrasound energy.

In certain embodiments Applicants' hand-held ultrasound device includes an “auto-detect” feature, wherein that devices monitors the reflected diagnostic signals, and automatically detects the arrival of the injected microbubbles at the occlusion site. When those injected microbubbles are detected, Applicants' device automatically causes the plurality of therapeutic ultrasound devices to emit therapeutic ultrasound energy. When the flow of microbubbles ceases, Applicants' device automatically causes the plurality of therapeutic ultrasound devices to stop emitting therapeutic ultrasound energy.

Referring to FIG. 8E, device 802 includes controller 895. Controller 895 includes a processor and device microcode to operate diagnostic transceiver 810 and each of the plurality of therapeutic ultrasound emitters. Controller 895 further includes microcode which processes the reflected signals provided by transceiver 810. Controller 895 is capable of detecting the arrival of the injected microbubbles at the occlusion site. Controller 895 causes one or more of therapeutic emitters to emit therapeutic ultrasound energy. When controller 895 detects the absence of microbubbles, controller 895 causes those therapeutic emitters to stop emitting sound waves.

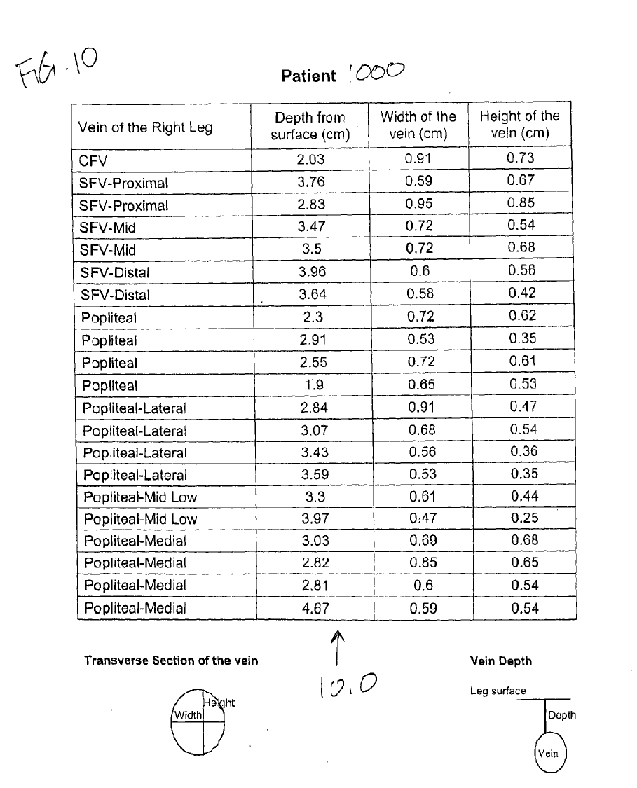

FIG. 9 summarizes Applicants' method to use the various embodiments of Applicants' hand-held ultrasonic device to treat an occlusion lodged in a blood vessel. In certain embodiments, the occluded vessel comprises an artery. In certain embodiments, the occluded vessel comprises a vein. In certain embodiments, the occluded vessel comprises an artery/vein disposed in a patient's leg. In certain embodiments, the occluded vessel comprises an artery/vein disposed in a patient's arm. In certain embodiments, the occluded vessel comprises an artery/vein disposed in a patient's myocardium. In certain embodiments, the occluded vessel comprises an artery/vein disposed within a patient's cranial cavity.

In step 905, the method provides an injectable microbubble formulation. U.S. Pat. Nos. 5,656,211 and 6,033,646 teach methods to form such a microbubble formulation, and are hereby incorporated by reference herein. U.S. Pat. No. 6,039,557 teaches an apparatus for preparing such a microbubble formulation, and is hereby incorporated by reference herein.

In step 910, the method determines the situs of the blood vessel occlusion. As those skilled in the art will appreciate, various methods exist to determine that situs. Step 910 includes identifying the occluded vessel. Step 910 further includes. identifying the location of the occlusion in that subject vessel. In certain embodiments, step 910 further includes determining the depth of the occluded vessel portion from the skin surface. In certain embodiments, step 910 further includes determining the width of the vessel at the occlusion. In certain embodiments, step 910 further includes determining the height of the vessel at the occlusion.

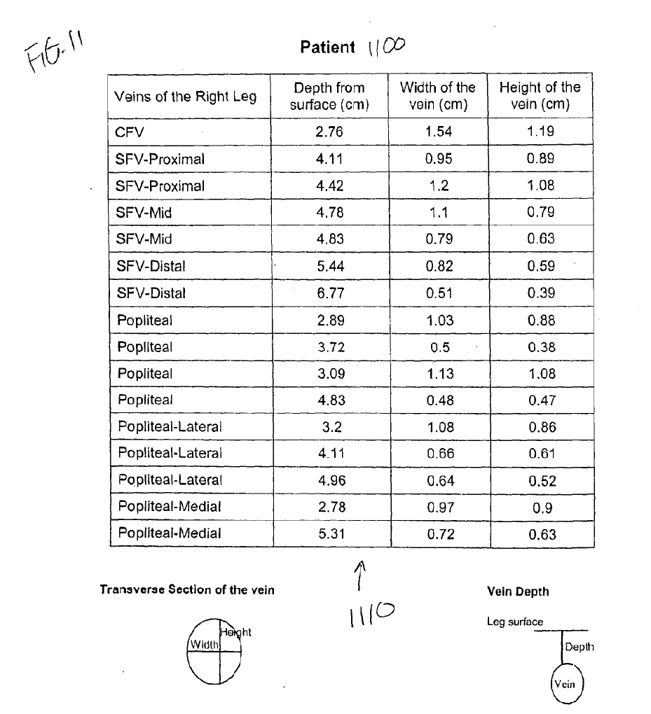

Referring to FIG. 10, chart 1010 shows measurement data for various veins disposed in the leg of a human patient 1000. Chart 1010 recites depth from surface data, vein width data, and vein height data.

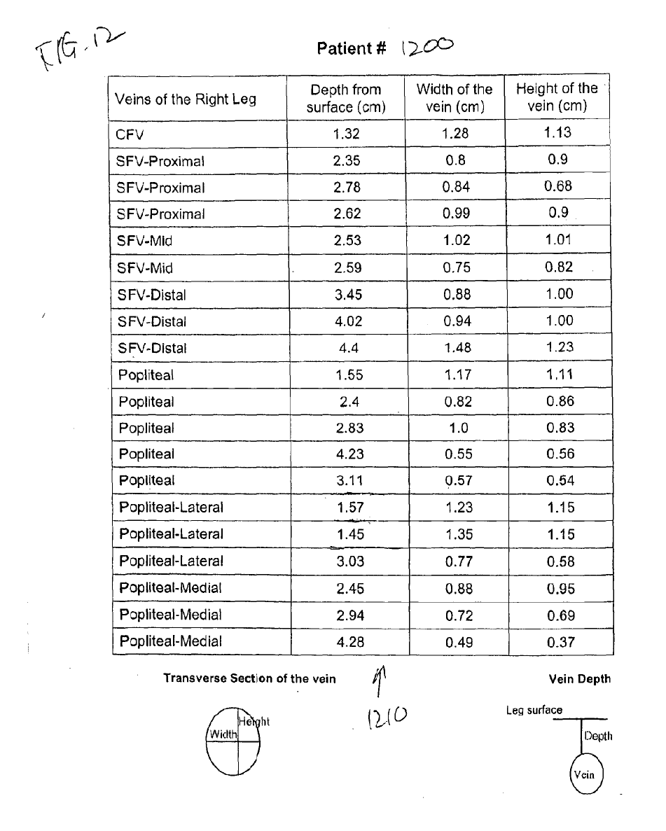

Referring to FIG. 11, chart 1110 shows measurement data for various veins disposed in the leg of a human patient 1100. Chart 1110 recites depth from surface data, vein width data, and vein height data.

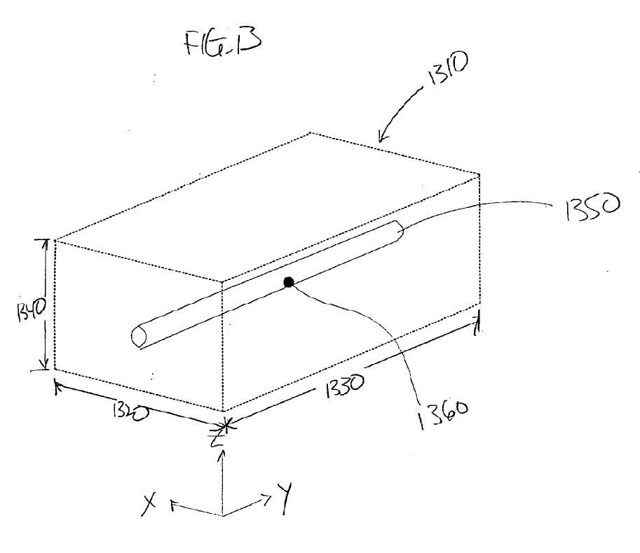

Referring to FIG. 12, chart 1210 shows measurement data for various veins disposed in the leg of a human patient 1200. Chart 1210 recites depth from surface data, vein width data, and vein height data.

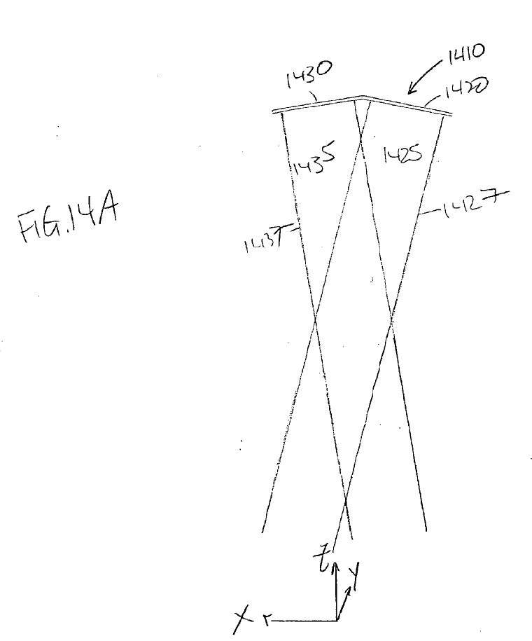

In step 915, the method selects a therapeutic ultrasound emitting device and power level based upon the determinations of step 910. Referring now to FIG. 13, vessel 1350 includes occlusion site 1360. Using the determinations of step 910, and estimating the error levels of those various determinations, the operator defines a target subcutaneous energy envelope 1310. Energy envelope 1310 includes dimension 1340 along the Z direction, dimension 1320 along the X direction, and dimension 1330 along the Y direction.

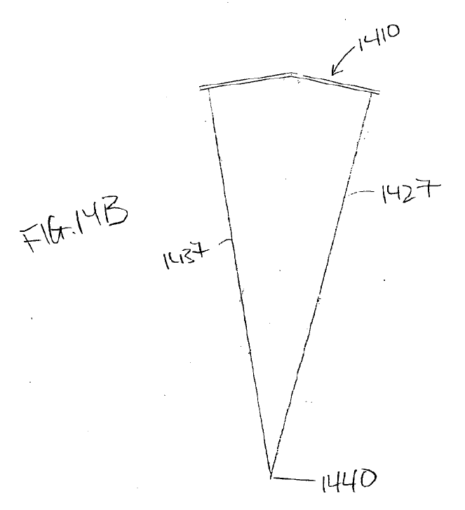

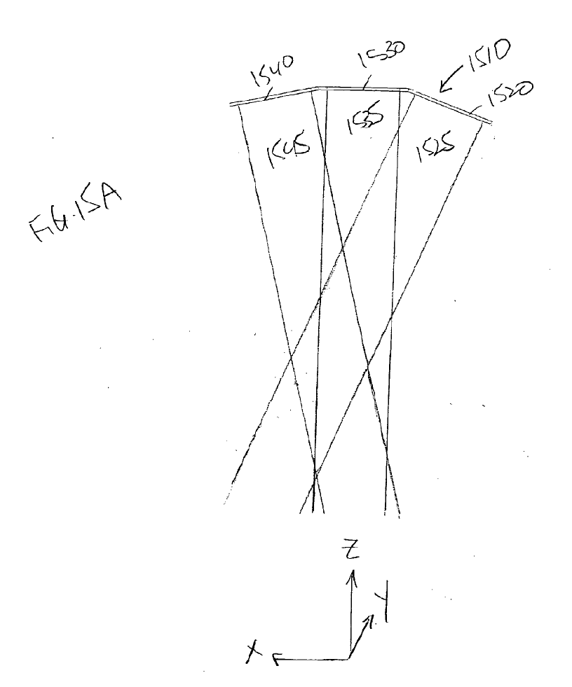

Having determined a target energy envelope, step 915 further includes selecting a sound head matrix that emits an actual ultrasound energy envelope that most closely corresponds to the desired target energy envelope. Step 915 further includes determining output power levels, and an emitter operating protocol, i.e. continuous or discontinuous operation. FIG. 14A shows a cross-sectional view of the ultrasound energy profile in the X/Z plane generated by a 2×Z offset sound head matrix 1410. First emitter 1420 produces energy profile 1425. Second emitter 1430 produces energy profile 1435. FIG. 14B shows convergence point 1440 for the overlapping energy profiles for emitter 1420 and 1430.

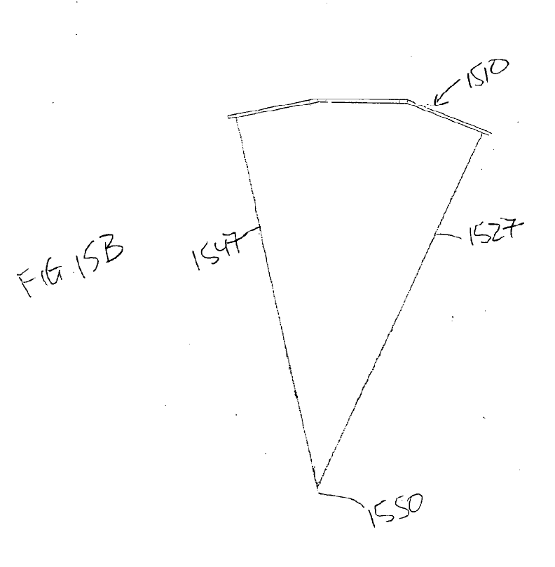

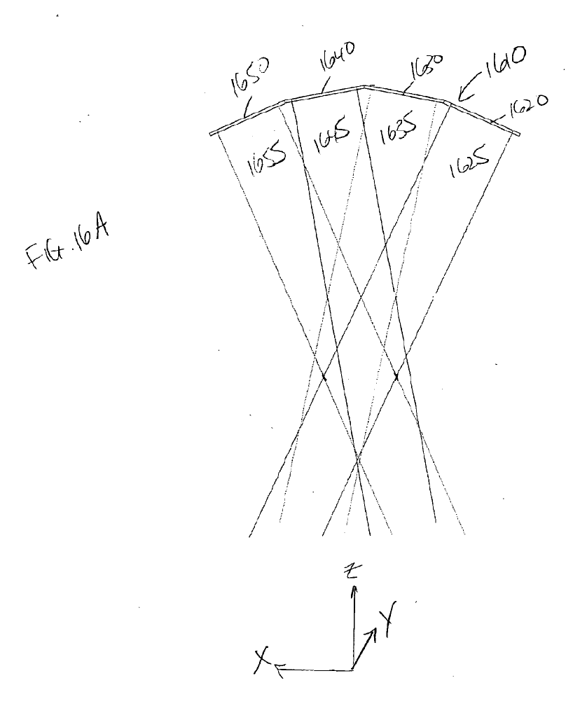

FIG. 15A shows a cross-sectional view of the ultrasound energy profile, in the X/Z plane, generated by a 3×Z offset sound head matrix 1510. First emitter 1520 produces energy profile 1525. Second emitter 1530 produces energy profile 1535. Third emitter 1540 produces energy profile 1545. FIG. 15B shows convergence point 1550 for the overlapping energy profiles for emitters 1520, 1530, and 1540.

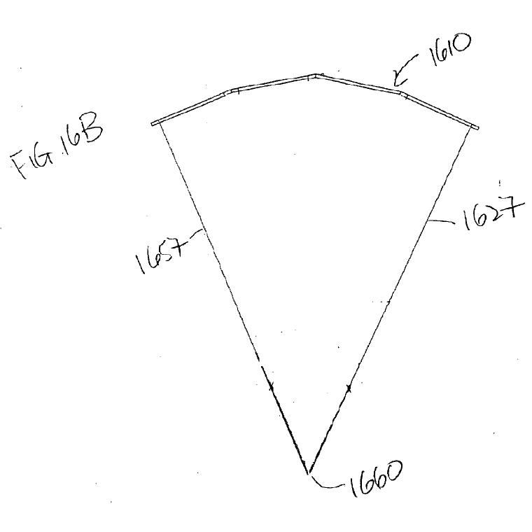

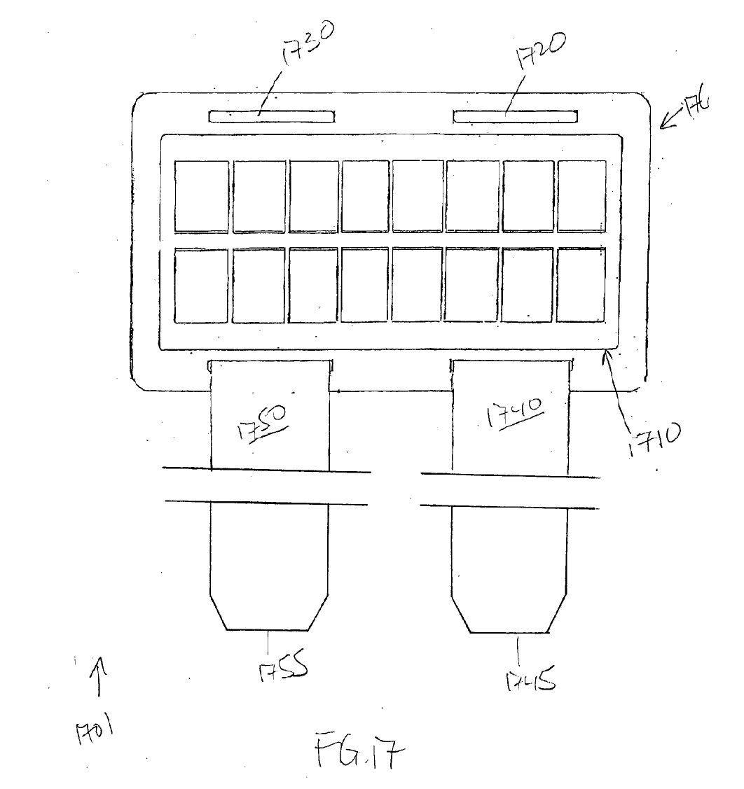

FIG. 16A shows a cross-sectional view of the ultrasound energy profile, in the X/Z plane, generated by a 4×Z offset sound head matrix 1610. First emitter 1620 produces energy profile 1625. Second emitter 1630 produces energy profile 1635. Third emitter 1640 produces energy profile 1645. Fourth emitter 1650 produces energy profile 1655. FIG. 16B shows convergence point 1660 for the overlapping energy profiles for emitters 1620, 1630, 1640, and 1650.

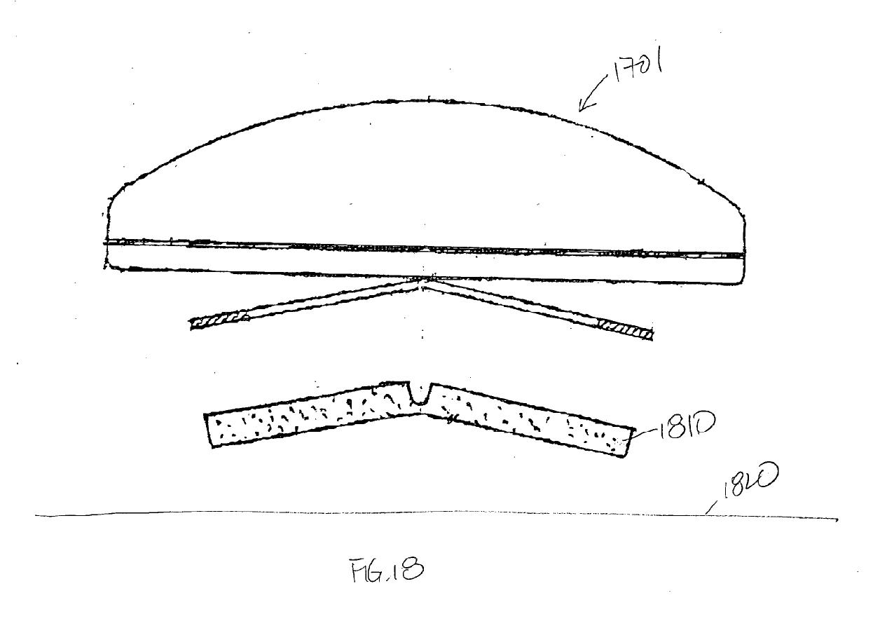

Referring again to FIG. 9, in step 920 Applicants' method positions the therapeutic ultrasound emitting device, selected in step 915, over the occlusion site located in step 910. Referring now to FIG. 17, Applicants' hand-held ultrasound device 1701 includes a 2×8 offset sound head matrix assembly 1710 and housing 1760 (FIG. 1C), where housing 1760 is formed to include apertures 1720 and 1730 extending through a first side of that housing. Device 1701 further includes elastic straps 1740 and 1750, where one end of those straps is attached to the second side of housing 1760 adjacent sound head matrix assembly 1710. The distal end of elastic strap 1740 comprises tab 1745. The distal end of elastic strap 1750 comprises tab 1755.

Device 1701 can be releaseably attached to the patient's extremity by advancing elastic straps 1740 and 1750 around that extremity, inserting tab 1745 into and through aperture 1720, securing tab 1745, inserting tab 1755 into and through aperture 1730, and securing tab 1755. In certain embodiments, tabs 1745 and 1755 are secured using hook and loop fasteners, i.e. VELCRO® fasteners. In other embodiments, tabs 1745 and 1755 are secured using buckle devices disposed on housing 1760.

Referring to FIG. 18, ultrasound coupling medium 1810 is positioned on skin surface 1820 over the occlusion site. Applicants' hand-held ultrasound device 1701 is then place on top coupling medium 1810. Device 1701 can then be secured in position using straps 1740 (FIG. 17) and 1750 (FIG. 17). In certain embodiments, the ultrasound coupling medium comprises carageenan. As those skilled in the art will appreciate, carageenan is a long chain polysacharide with a backbone of the sugar galactose. In other embodiments, the ultrasound coupling medium comprises xanthum gum. In other embodiments, the ultrasound coupling medium comprises alginic acid. As those skilled in the art will appreciate, alginic acid is a naturally occurring hydrophilic colloidal polysaccharide obtained from the various species of brown seaweed (Phaeophyceae), and comprises a linear copolymer consisting mainly of residues of b-1,4-linked D-mannuronic acid and a-1,4-linked L-glucuronic acid. In other embodiments, the ultrasound coupling medium comprises a silicon gel.

Referring again to FIG. 9, after positioning the selected therapeutic ultrasound emitting device over the occlusion site in step 920, Applicants' method transitions to step 925 wherein the medical provider injects the microbubble formulation of step 905 into the subject vessel distal to the occlusion site.

In step 930, Applicants' method determines if the ultrasound device selected in step 915 includes a diagnostic emitter. If Applicants' method determines in step 930 that the selected hand-held ultrasound device includes a diagnostic ultrasound emitter, then Applicant's method transitions to step 955 wherein the method determines if the selected device includes an auto-detect function. If Applicants' method determines in step 955 that the device selected in step 915 includes both a diagnostic ultrasound emitter and an auto-detect function, then Applicants' method transitions from step 955 to step 960 wherein the operator initiates the auto-detect function. In embodiments wherein the selected device includes both a diagnostic ultrasound emitter and an auto-detect function, the operator need do no more than initiate the auto-detect function. The apparatus then automatically detects the arrival of the microbubbles at the occlusion site, automatically initiates the selected ultrasound emission program, automatically detects the absence of microbubbles at the occlusion site, and automatically discontinues ultrasound emissions.

If Applicants' method determines in step 955 that the selected device does not include an auto-detect function, then the method transitions from step 955 to step 965 wherein the operator determines if the selected device includes a display screen in combination with the diagnostic ultrasound emitter. If the selected device does include a display screen in combination with the diagnostic ultrasound emitter, then the method transitions to step 975 wherein the operator monitors the display device.

In step 980, the operator visually sees, using the display device, the presence of microbubbles at the occlusion site. Applicants' method transitions from step 980 to step 985 wherein the operator causes the hand-held ultrasound device to provide therapeutic ultrasound energy to the occlusion site. In step 990, the operator visually detects the absence of microbubbles at the occlusion site. Applicants' method transitions from step 990 to step 950 wherein the operator discontinues ultrasound emissions.

If the operator determines in step 965 that the selected hand-held ultrasound device includes a diagnostic ultrasound emitter but does not include a display screen, then the method transitions from step 965 to step 970 wherein the operator receives an indication that microbubbles are present at the occlusion site. In certain embodiments, the indication of step 970 comprises a visual alert, such as for example a flashing light. In certain embodiments, the indication of step 970 comprises a auditory alert. Applicants' method transitions from step 970 to step 935 wherein the operator determines a treatment time interval. That treatment time interval comprises an estimate made by the operator of the time period in which microbubbles are likely to be present at the occlusion site. Applicants' method transitions from step 935 to step 940 wherein the operator causes the selected device to emit therapeutic ultrasound energy. In certain embodiments, steps 935 and 940 are performed substantially synchronously.

In step 945, the operator determines if the treatment time interval selected in step 935 has expired. If the operator determines that the treatment time interval has not expired, then the method continues to provide therapeutic ultrasound energy to the occlusion site. Alternatively, if the operator determines in step 945 that the treatment time interval has expired, then the method transitions from step 945 to step 950 wherein the operator discontinues ultrasound emissions.

In certain embodiments, individual steps recited in FIG. 3A, and/or FIG. 3B, and/or FIG. 3C, may be combined, eliminated, or reordered.

In certain embodiments, Applicants' invention includes microcode, such as microcode 626, where that microcode is executed by a controller, such as controller 620 (FIG. 6)/720 (FIGS. 7A, 7B)/805 (FIGS. 8B, 8C), 895 (FIG. 8E), to perform one or more of steps 935, 940, 945, 950, 960, 980, 985, 990, recited in FIG. 9.

In other embodiments, Applicants' invention includes instructions residing in any other computer program product, where those instructions are executed by a computer external to, or internal to, Applicants' hand-held apparatus to perform steps one or more of steps 935, 940, 945, 950, 960, 980, 985, 990, recited in FIG. 9. In either case, the microcode/instructions may be encoded in an information storage medium comprising, for example, a magnetic information storage medium, an optical information storage medium, an electronic information storage medium, and the like. By “electronic storage media,” Applicants mean, for example, a device such as a PROM, EPROM, EEPROM, Flash PROM, compactflash, smartmedia, and the like.

While the preferred embodiments of the present invention have been illustrated in detail, it should be apparent that modifications and adaptations to those embodiments may occur to one skilled in the art without departing from the scope of the present invention.

We claim:

1. An ultrasound energy emitting apparatus, comprising:
a hand-held enclosure;
a plurality of ultrasound transducers disposed on said enclosure.

2. The apparatus of claim 1, wherein said plurality of ultrasound transducers are disposed within said enclosure and extend outwardly through said enclosure.

3. The apparatus of claim 1, wherein said plurality of ultrasound transducers comprises a sound head matrix comprising (M) rows of ultrasound transducers and (N) columns of ultrasound transducers.

4. The apparatus of claim 3, wherein (M) is greater than or equal to 2, and wherein (N) is greater than or equal to 2

5. The apparatus of claim 3, wherein said enclosure comprises (N) planar members, wherein (M) ultrasound transducers are disposed on, or extend through, each of said (N) planar members.

6. The apparatus of claim 5, wherein the (i)th planar member is attached to at least one of the (i+1)th planar member and the (i−1)th planar member, wherein (i) is greater than or equal to 1, and less than or equal to (N).

7. The apparatus of claim 5, wherein the (i)th planar member in combination with the (i−1)th planar member define an interior dihedral angle less than 180 degrees.

8. The apparatus of claim 7, wherein the (i)th planar member is moveably attached to said (i−1)th planar member such that said interior dihedral angle can be adjusted from about 90 degrees to about 180 degrees.

9. The apparatus of claim 3, further comprising:
a controller;
a plurality of communication links, wherein a different one of said plurality of communication links interconnects a different one of said plurality of ultrasonic transducers to said controller.

10. The apparatus of claim 9, further comprising device microcode, wherein said controller using said device microcode is capable of causing one or more of, but fewer than all of, said plurality of ultrasound transducers to emit ultrasound energy.

11. The apparatus of claim 9, wherein said controller is disposed within said enclosure.

12. The apparatus of claim 9, further comprising a visual display device interconnected to said controller.

13. The apparatus of claim 12, further comprising a diagnostic ultrasound transceiver, wherein said diagnostic ultrasound transceiver is interconnected with said controller and with said visual display device.

14. A method to treat a patient having an occlusion lodged in a blood vessel, comprising the steps of:
locating an occlusion site in an occluded blood vessel;
supplying an ultrasound emitting apparatus comprising a sound head matrix comprising (M) rows of ultrasound transducers and (N) columns of ultrasound transducers, wherein said (M) ultrasound transducers are disposed on, or extend through, (N) planar members, wherein the (i)th planar member is attached to at least one of the (i+1)th planar member and the (i−1)th planar member, wherein (i) is greater than or equal to 1, and less than or equal to (N), and wherein the (i)th planar member in combination with the (i+1)th planar member define the (i)th interior dihedral angle, wherein said (i)th dihedral angle is less than 180 degrees;
supplying an injectable microbubble formulation;
positioning said ultrasound emitting device over said occlusion site;
injecting said injectable microbubble formulation into said occluded blood vessel distal to said occlusion site; and
providing to said occlusion site ultrasound energy from said ultrasound emitting device.

15. The method of claim 14, further comprising the steps of:
emitting ultrasound energy from the (M) ultrasound transducers disposed on the (i)th planar member;
emitting second ultrasound energy from the (M) ultrasound transducers disposed on the (i+1)th planar member;
overlapping said first ultrasound energy and said second ultrasound energy at a convergence point.

16. The method of claim 15, further comprising the step of adjusting the (i)th interior dihedral angle to adjust the location of said convergence point.

17. The method of claim 14, wherein (M) is greater than or equal to 2, and wherein said providing step further comprises:
causing the ultrasound transducers arranged in a first row of said sound head matrix to emit ultrasound energy during a first time interval;
causing the ultrasound transducers arranged in a second row of said sound head matrix to emit ultrasound energy during a second time interval, wherein said first time interval differs from said second time interval.

18. The method of claim 17, further comprising the steps of:
defining a treatment duration;
alternating said first time interval and said second time interval throughout said treatment duration.

19. The method of claim 14, wherein (N) is greater than or equal to 2, and wherein said providing step further comprises:
causing the ultrasound transducers arranged in a first column of said sound head matrix to emit ultrasound energy during a first time interval;
causing the ultrasound transducers in a second column of said sound head matrix to emit ultrasound energy during a second time interval, wherein said first column differs from said second column, and wherein said first time interval differs from said second time interval.

20. The method of claim 19, further comprising the steps of:
defining a treatment duration;
alternating said first time interval and said second time interval throughout said treatment duration.

21. The method of claim 14, wherein said supplying an ultrasound emitting device step further comprises supplying an ultrasound emitting device comprising a diagnostic ultrasound transceiver interconnected to a visual display device, and wherein said method further comprises the step of visually detecting the arrival of said microbubbles at said occlusion step using said visual display device.

22. The method of claim 14, wherein said supplying an ultrasound emitting device step further comprises supplying an ultrasound emitting device comprising a diagnostic ultrasound transceiver and an auto-detect function, and wherein said method further comprises the steps of:
initiating said auto-detect mode; and
detecting by said diagnostic ultrasound transceiver the arrival of said microbubbles at said occlusion site.

23. An article of manufacture comprising a plurality of ultrasound transducers and a computer useable medium having computer readable program code disposed therein to operate said plurality of ultrasound transducers, the computer readable program code comprising a series of computer readable program steps to effect causing said plurality of ultrasound transducers to emit ultrasound energy.

24. The article of manufacture of step 23, said computer readable program code further comprising a series of computer readable program steps to effect causing one or more of, but fewer than all of, said plurality of ultrasound transducers to emit ultrasound energy.

25. The article of manufacture of claim 24, wherein said plurality of ultrasound transducers comprises two or more rows of ultrasound transducers and two or more columns of ultrasound transducers, said computer readable program code further comprising a series of computer readable program steps to effect:
causing the ultrasound transducers arranged in a first row of said sound head matrix to emit ultrasound energy during a first time interval;
causing the ultrasound transducers arranged in a second row of said sound head matrix to emit ultrasound energy during a second time interval, wherein said first time interval differs from said second time interval.

26. The article of manufacture of step 25, said computer readable program code further comprising a series of computer readable program steps to effect:
retrieving a predetermined treatment time duration;
alternating said first time interval and said second time interval throughout said treatment time duration.

27. The article of manufacture of claim 24, wherein said plurality of ultrasound transducers comprises two or more rows of ultrasound transducers and two or more columns of ultrasound transducers, said computer readable program code further comprising a series of computer readable program steps to effect:
causing the ultrasound transducers arranged in a first column of said sound head matrix to emit ultrasound energy during a first time interval;
causing the ultrasound transducers arranged in a second column of said sound head matrix to emit ultrasound energy during a second time interval, wherein said first time interval differs from said second time interval.

28. The article of manufacture of step 27, said computer readable program code further comprising a series of computer readable program steps to effect:
retrieving a predetermined treatment time duration;
alternating said first time interval and said second time interval throughout said treatment time duration.

29. The article of manufacture of step 23, further comprising a diagnostic ultrasound transceiver and a visual display device, said computer readable program code further comprising a series of computer readable program steps to effect:
emitting diagnostic ultrasound energy from said diagnostic ultrasound transceiver;
receiving reflected emissions;
displaying an image of tissue structures underlying said diagnostic ultrasound transceiver.

30. A computer program product usable with a programmable computer processor to operate a hand-held apparatus comprising a plurality of ultrasound transducers, comprising computer readable program code which causes said programmable computer processor to energize said plurality of ultrasound transducers thereby causing said plurality of ultrasound transducers to emit ultrasound energy.

31. The computer program product of step 30, further comprising computer readable program code which causes said programmable computer processor to cause one or more of, but fewer than all of, said plurality of ultrasound transducers to emit ultrasound energy.

32. The computer program product of claim 31, wherein said plurality of ultrasound transducers comprises two or more rows of ultrasound transducers and two or more columns of ultrasound transducers, further comprising:
computer readable program code which causes said programmable computer processor to cause the ultrasound transducers arranged in a first row of said sound head matrix to emit ultrasound energy during a first time interval;
computer readable program code which causes said programmable computer processor to cause the ultrasound transducers arranged in a second row of said sound head matrix to emit ultrasound energy during a second time interval, wherein said first time interval differs from said second time interval.

33. The computer program product of step 32, further comprising:
computer readable program code which causes said programmable computer processor to retrieve a predetermined treatment time duration;
computer readable program code which causes said programmable computer processor to alternate said first time interval and said second time interval throughout said treatment time duration.

34. The computer program product of claim 3 1, wherein said plurality of ultrasound transducers comprises two or more rows of ultrasound transducers and two or more columns of ultrasound transducers, further comprising:
computer readable program code which causes said programmable computer processor to cause the ultrasound transducers arranged in a first column of said sound head matrix to emit ultrasound energy during a first time interval;
computer readable program code which causes said programmable computer processor to cause the ultrasound transducers arranged in a second column of said sound head matrix to emit ultrasound energy during a second time interval, wherein said first time interval differs from said second time interval.

35. The computer program product of step 34, said computer readable program code further comprising a series of computer readable program steps to effect:
computer readable program code which causes said programmable computer processor to retrieve a predetermined treatment time duration;
computer readable program code which causes said programmable computer processor to alternate said first time interval and said second time interval throughout said treatment time duration.

36. The computer program product of step 30, wherein said hand-held apparatus further comprises a diagnostic ultrasound transceiver and a visual display device, further comprising:
computer readable program code which causes said programmable computer processor to emit diagnostic ultrasound energy from said diagnostic ultrasound transceiver;
computer readable program code which causes said programmable computer processor to receive reflected emissions;
computer readable program code which causes said programmable computer processor to display an image of tissue structures underlying said diagnostic ultrasound transceiver.
